(19) United States
(12) Patent Application Publication
(10) Pub. No.: US 2026/0053348 A1
(43) Pub. Date: Feb. 26, 2026

(54) SYSTEMS AND METHODS FOR DETECTING AND TREATING EYE MISALIGNMENT WITH DYNAMIC TASKS

(71) Applicant: Zenni Optical, Inc., Novato, CA (US)

(72) Inventors: Steven LEE, Barrington, IL (US); Julia ZHEN, Novato, CA (US); ChyrSong TING, Novato, CA (US); Matthew James GOLINO, Brookhaven, GA (US); Justin Paul DEMPSEY, Ottawa (CA); Jeffrey Joseph FILLINGHAM, Dartmouth (CA)

(21) Appl. No.: 18/811,683

(22) Filed: Aug. 21, 2024

(51) Int. Cl. A61B 3/08; A61B 3/00

(52) U.S. Cl. CPC A61B 3/085; A61B 3/0025; A61B 3/005

(57) ABSTRACT

A patient's visual health can be evaluated via virtual reality (VR) system in electronic communication with a computing device. The computing device can cause a first visual task to be displayed on the VR system. The VR system can collect the patient's responses to the first visual task, and the computing device can analyze the patient's responses to develop a second visual task to be displayed on the VR system. This procedure may continue until the computing device has completed a comprehensive evaluation of the patient's eye coordination and eye misalignment. Optionally, the computing device can cause the VR system to deliver corrective feedback to the patient. The computing device can diagnose the patient with one or more ocular conditions and recommend treatment.

### BACKGROUND

### Field of the Inventions

The present application relates to methods of assessing various ocular conditions through extended reality systems. More specifically, methods and systems are applied to conduct visual tasks and exams in extended reality environments to evaluate patients for ocular conditions and diseases, such as eye misalignment and visual processing disorders.

### Description of the Related Art

As virtual reality (VR) technology has become increasingly sophisticated, new highly immersive experiences have been made possible through improvements in head and motion tracking systems. Eye-tracking technology allows systems to detect and respond to where the user is looking. This capability enhances user interaction and makes virtual environments more responsive and engaging. Eye tracking is being integrated into a variety of VR applications, from gaming and training simulations to medical diagnostics and research, as it offers a more intuitive way for users to interact with digital content.

### SUMMARY

The systems, methods and devices of this disclosure each have several innovative aspects, no single one of which is solely responsible for the desirable attributes disclosed herein.

Despite the advancements in VR technology, and in particular, eye-tracking technology, in accordance with some embodiments disclosed herein is the realization that VR technology can provide unique eyecare solutions through monitoring and tracking one or more of the user's eyes and providing a diagnostic, treatment protocol, and/or treatment system. Indeed, in accordance with some embodiments disclosed herein is the realization that VR technology can be used to address challenges associated with diagnosing a variety of eye disorders and ocular conditions, such as detecting misalignment, macular degeneration, tear film characteristics, floater characteristics, eye tracking issues, motion sensitivity, and other eye movement disorders and the treatment of such.

One method of this disclosure is a virtual reality (VR) method designed to simulate a range of visual disturbances, thereby assisting in the diagnosis of ocular conditions. This method leverages advanced VR technology to create an immersive and controlled environment where users experience various visual anomalies such as blurriness, double vision, floaters, and field loss. By mimicking these visual disturbances in a highly realistic manner, this method and system aid eye care professionals in identifying and differentiating between various ocular pathologies, including but not limited to cataracts, glaucoma, macular degeneration, and retinal detachment. The method can be carried out on a system that includes a VR headset or other head-mounted display integrated with eye-tracking sensors and real-time rendering software. These components work synergistically to provide accurate and customizable simulations tailored to the specific symptoms reported by patients.

The system for this first method features implementation of a machine learning algorithm that adapts the simulated visual disturbances in real-time based on the patient's ocular response and feedback. This algorithm utilizes data and feedback collected from the eye-tracking sensors to dynamically adjust the intensity, frequency, and nature of the visual anomalies presented to the user. Moreover, the system includes a proprietary diagnostic module that cross-references the patient's interactive experience with a comprehensive database of ocular conditions, providing predictive insights and suggesting potential diagnoses with high accuracy. The combination of real-time adaptive simulation and predictive diagnostics offers a personalized and highly precise tool for early detection and treatment planning of ocular diseases. This not only enhances diagnostic accuracy but also reduces the need for invasive procedures and prolonged testing, positioning it as a groundbreaking advancement in eye care technology.

A second method of this disclosure is a VR method specifically designed for the screening of eye diseases using symptom-specific visual tests combined with artificial intelligence (AI) analysis. This innovative method employs a VR headset equipped with high-resolution displays and integrated eye-tracking technology to present a series of tailored visual tests that simulate various ocular symptoms. These tests are designed to assess visual acuity, field of vision, contrast sensitivity, color perception, and other critical visual functions. The system records the patient's responses and eye movements during the tests, providing comprehensive data that eye care professionals can use to identify potential eye diseases such as diabetic retinopathy, glaucoma, macular degeneration, and refractive errors. This approach offers a non-invasive, efficient, and engaging way to screen for eye conditions, making it accessible for routine use in both clinical and home settings.

The system for this second method integrates an advanced AI-driven diagnostic engine that analyzes the collected data in real-time to generate predictive models of ocular health. This diagnostic engine leverages deep learning algorithms trained on a vast dataset of visual test responses and known diagnoses to identify subtle patterns and correlations that may not be apparent through traditional screening methods. This system adapts the visual tests dynamically based on the real-time analysis of the patient's performance. For example, if the AI detects signs of early-stage glaucoma based on the patient's visual field test results, it can immediately adjust subsequent tests to focus more intensively on detecting glaucoma-specific symptoms, thereby increasing diagnostic accuracy. Additionally, the system includes a feedback mechanism that provides personalized recommendations for further testing or treatment based on the AI's findings, which is tailored to the individual's risk profile and ocular health history. This sophisticated combination of adaptive testing and AI-driven diagnostics represents a significant leap forward in the early detection and management of eye diseases, offering a highly personalized and precise screening tool that is likely to be patentable due to its innovative and nuanced approach.

A third method of this disclosure is a VR method for detecting eye misalignment by engaging users in tasks that require precise eye coordination. This technique utilizes a VR headset equipped with high-fidelity displays and sophisticated eye-tracking sensors to create an immersive environment where users perform various visual tasks that challenge their binocular vision. These tasks include depth perception exercises, alignment of virtual objects, and real-time tracking of moving targets, all of which are meticulously designed to assess the coordination between both eyes. The VR system captures detailed data on eye movements, pupil responses, and fixation stability, providing critical insights into the presence and severity of eye misalignment conditions such as strabismus, amblyopia, and convergence insufficiency. This method offers a non-invasive, engaging, and highly accurate approach to diagnosing eye misalignment, making it suitable for use in clinical, educational, and home settings.

Furthermore, the system for this third method utilizes a multi-layered, adaptive diagnostic algorithm that not only evaluates eye coordination but also provides real-time corrective feedback through haptic and visual stimuli. This algorithm dynamically adjusts the difficulty and nature of the visual tasks based on the user's performance, thereby personalizing the assessment to the individual's specific condition. Furthermore, the system that utilizes machine learning to predict the likelihood of future misalignment issues based on current eye coordination patterns and historical data. By analyzing subtle discrepancies in eye movements and predicting potential misalignment trends, the VR technique can offer preemptive therapeutic exercises tailored to strengthen binocular vision and prevent the progression of misalignment. This predictive and corrective capability, combined with the adaptive task adjustment, represents a significant innovation in the field of ophthalmology. It enhances the diagnostic process and provides a proactive approach to managing eye alignment issues, offering a compelling case for patentability due to its originality and practical application.

A fourth method of this disclosure is a VR method for the vision screening of children through the use of interactive games. This system leverages VR technology to create an engaging and immersive environment where children can participate in a series of gamified visual tests. These tests are carefully crafted to assess various aspects of visual health, including visual acuity, depth perception, color vision, and eye coordination. The VR headset, equipped with high-resolution displays and precise eye-tracking sensors, monitors the child's eye movements and visual responses during gameplay. By transforming traditional vision screening into a fun and interactive experience, this VR solution not only captures the child's attention but also reduces anxiety and improves compliance with the screening process.

Moreover, the system for the fourth method of vision evaluation incorporates an advanced adaptive learning system that tailors the difficulty and type of visual tasks based on the child's age, developmental stage, and real-time performance. This system utilizes AI to analyze the child's responses and dynamically adjust the gameplay to maintain an optimal challenge level, ensuring accurate and reliable assessment results. Additionally, the VR solution uses biometric feedback, such as heart rate and pupil dilation, to gauge the child's engagement and stress levels during the screening. By combining these biometric indicators with eye-tracking data, the system can identify instances where the child's performance may be influenced by external factors, thus enhancing the precision of the diagnostic outcomes. Specifically, the data collected through these interactive games provide valuable insights for diagnosing common pediatric vision problems such as amblyopia, strabismus, and refractive errors. Furthermore, the solution includes a unique parental interface that provides detailed reports on the child's visual health, along with personalized recommendations for further testing or corrective measures. This combination of adaptive learning, biometric feedback, and detailed parental reporting represents a significant advancement in pediatric vision screening, offering a highly personalized and effective tool for early detection and intervention of vision problems in children.

Finally, fifth method of this disclosure is a VR method for detecting potential visual processing disorders using multisensory integration. This method leverages the immersive capabilities of a VR system to create a controlled environment where users are exposed to various sensory stimuli, including visual, auditory, and tactile inputs. The system employs a VR headset equipped with high-resolution displays and advanced eye-tracking sensors, along with synchronized auditory and haptic feedback devices, to present a series of interactive tasks. These tasks are designed to assess the user's ability to process and integrate sensory information, focusing on identifying discrepancies in visual perception and response. By monitoring the user's eye movements, reaction times, and coordination across multiple sensory modalities, the application provides comprehensive data that can help diagnose visual processing disorders such as dyslexia, visual-motor integration issues, and other related conditions.

The system for the fifth method of vision evaluation implements a real-time sensory adaptation algorithm that adjusts the intensity and nature of the multisensory stimuli based on the user's performance and physiological responses. This algorithm employs machine learning techniques to analyze data from eye-tracking sensors, heart rate monitors, and skin conductance sensors to dynamically modify the sensory inputs. For instance, if the system detects that the user is struggling with visual stimuli, it can enhance auditory or tactile feedback to support the visual processing task, thereby creating a balanced sensory environment tailored to the individual's needs. Additionally, the application includes a novel diagnostic module that cross-references the user's multisensory integration patterns with a database of known visual processing disorders, providing predictive insights and personalized recommendations for therapeutic interventions. This capability to adaptively tailor sensory experiences and provide predictive diagnostics based on multisensory data represents a significant advancement in the field of neuro-ophthalmology, offering a powerful tool for early detection and personalized treatment of visual processing disorders.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 is an example data processing environment having one or more servers communicatively coupled to one or more computer devices, in accordance with some embodiments.

FIG. 2 is an environment in which a computer device is applied to facilitate visual assessment or eyewear fitting, in accordance with some embodiments.

FIG. 3 is a block diagram of a computer system configured to implement vision assessment or eyewear fitting, in accordance with some embodiments.

FIG. 4 is a block diagram of a machine learning system for training and applying machine learning models, in accordance with some embodiments.

FIG. 5A is a structural diagram of an example neural network applied to process input data in a machine learning model, in accordance with some embodiments.

FIG. 5B is an example node in the neural network, in accordance with some embodiments.

FIG. 6A is an example “tumbling E” chart applied in a visual acuity test, in accordance with some embodiments.

FIGS. 6B, 6C, 6D, and 6E are example patterns applied in an astigmatism test, a stereopsis test, a visual field test, and a color blindness test, in accordance with some embodiments.

FIG. 7 is another example visual pattern applied to test visual acuity and astigmatism, in accordance with some embodiments.

FIGS. 8A-8D include four diagrams of example graphical user interfaces rendered to determine a visual acuity score in a virtual environment created by a headset device, in accordance with some embodiments.

FIGS. 9A-9C include three diagrams of example graphical user interfaces rendered to determine a nearsighted or farsighted power in a virtual environment created by a headset device, in accordance with some embodiments.

FIGS. 10A-10F include six diagrams of example graphical user interfaces rendered to determine eye stigmatism in a virtual environment created by a headset device, in accordance with some embodiments.

FIGS. 11A and 11B illustrate the components of the vision evaluation system: the VR system and the computing device, in accordance with some embodiments.

FIGS. 12A-12D illustrate examples of different visual anomalies with varying degrees of intensity being displayed in the environment on the screen of the VR headset, in accordance with some embodiments.

FIGS. 13A-13D illustrate an example of the method of dynamically administering vision tests, in accordance with some embodiments.

FIG. 14A illustrates an example of the method of dynamically administering visual tasks to detect eye misalignment, in accordance with some embodiments.

FIG. 14B illustrates an example of the method of providing corrective feedback to a patient during visual tasks to correct eye misalignment, in accordance with some embodiments.

FIGS. 15A-15B illustrate a traditional method of administering a visual test and a gamified method of administering the visual test for children, in accordance with some embodiments.

FIG. 16 illustrates the relationship between the correctness of the patient's responses to a visual task, the intensity of the stimuli provided in the immersive VR environment during the visual task, and the patient's level of engagement during the visual task, in accordance with some embodiments.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

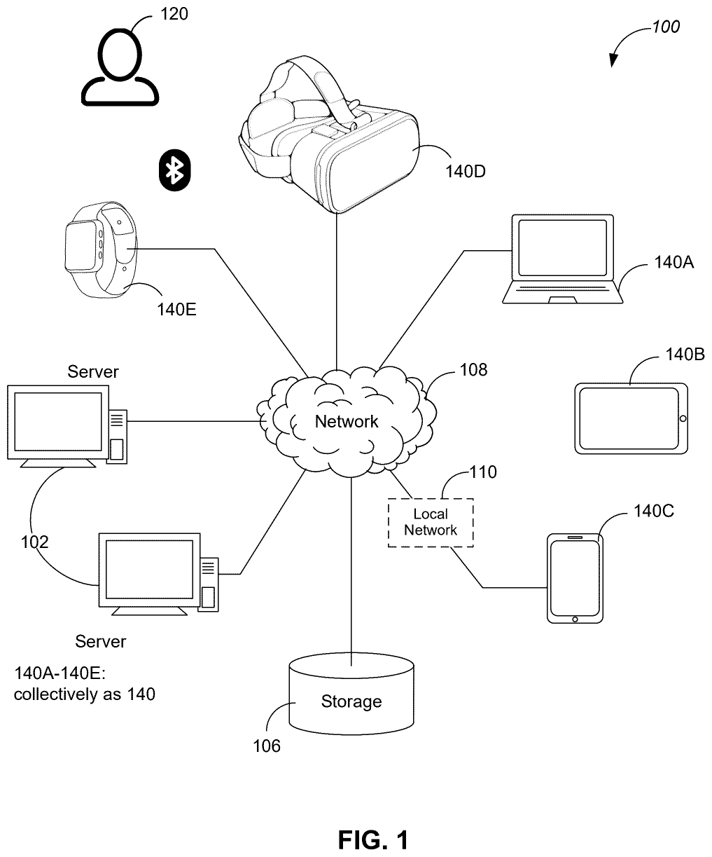

Referring now to the figures, FIG. 1 is an example data processing environment 100 having one or more servers 102 communicatively coupled to one or more computer devices 140 (e.g., a headset device 140D), in accordance with some embodiments. The one or more computer devices 140 are electronic devices having computational capabilities, and may be, for example, desktop computers 140A, tablet computers 140B, mobile phones 140C, or intelligent, multi-sensing, network-connected home devices (e.g., a depth camera, a visible light camera).

In some implementations, the one or more computer devices 140 can include a headset device 140D (also called a head-mounted display (HMD) device 140D) configured to render extended reality content. In some implementations, the one or more computer devices 140 can include a wireless wearable device 140E (e.g., a smart watch, a fitness band) configured to track health data (e.g., heart rate, quality of sleep) and activity data (e.g., steps walked, stairs climbed) of a user wearing the device 140E. Each computer device 140 can collect data or user inputs, executes user applications, and present outputs on its user interface. The collected data or user inputs can be processed locally at the computer device 140 and/or remotely by the server(s) 102. The one or more servers 102 can provide system data (e.g., boot files, operating system images, and user applications) to the computer devices 140, and in some embodiments, processes the data and user inputs received from the computer device(s) 140 when the user applications are executed on the computer devices 140. In some embodiments, the data processing environment 100 can further include a storage 106 for storing data related to the servers 102, computer devices 140, and applications executed on the computer devices 140. For example, storage 106 may store video content, static visual content, and/or audio data.

The one or more servers 102 can enable real-time data communication with the computer devices 140 that can be remote from each other or from the one or more servers 102. Further, in some embodiments, the one or more servers 102 can implement data processing tasks that are not completed locally by the computer devices 140. For example, the computer devices 140 can include a game console (e.g., the headset device 140D) that executes an interactive online gaming application (e.g., for visual assessment or eyewear fitting). The game console receives a user instruction and sends it to a server 102 with user data. The server 102 generates a stream of video data based on the user instruction and user data and provides the stream of video data for display on the game console and other computer devices that can be engaged in the same session with the game console.

The one or more servers 102, one or more computer devices 140, and storage 106 can be communicatively coupled to each other via one or more communication networks 108, which are the medium used to provide communications links between these devices and computers connected together within the data processing environment 100. The one or more communication networks 108 may include connections, such as wire, wireless communication links, or fiber optic cables. Examples of the one or more communication networks 108 include local area networks (LAN), wide area networks (WAN) such as the Internet, or a combination thereof. The one or more communication networks 108 are, optionally, implemented using any known network protocol includes various wired or wireless protocols, such as Ethernet, Universal Serial Bus (USB), FIREWIRE, Long Term Evolution (LTE), Global System for Mobile Communications (GSM), Enhanced Data GSM Environment (EDGE), code division multiple access (CDMA), time division multiple access (TDMA), Bluetooth, Wi-Fi, voice over Internet Protocol (VoIP), Wi-MAX, or any other suitable communication protocol. A connection to the one or more communication networks 108 may be established either directly (e.g., using 1G/4G connectivity to a wireless carrier), or through a network interface 110 (e.g., using a router, switch, gateway, hub, or an intelligent, dedicated whole-home control node), or through any combination thereof. As such, the one or more communication networks 108 can represent the Internet of a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other electronic systems that route data and messages.

In some embodiments, the headset device 140D can be communicatively coupled to a data processing environment 100. The headset device 140D includes one or more cameras (e.g., a visible light camera, a depth camera), a microphone, a speaker, one or more inertial sensors (e.g., gyroscope, accelerometer), and a display. In some embodiments, the camera may capture hand gestures of a user wearing the headset device 140D. In some embodiments, the microphone records ambient sound includes user's voice commands.

In some embodiments, the headset device 140D may be communicatively coupled to one or more servers 102 and enables a centralized vision test management platform with the one or more servers 102. This vision test management platform may aggregate data (e.g., visual stimuli 338, sensor data 342, vision test results 344) from a plurality of user accounts associated with a plurality of users, analyze the aggregated data, and track vision health trends for individual users or user groups. In some embodiments, data may be communicated between a headset device 140D and a server 102 in an encrypted format. In some embodiments, the vision test management platform is coupled to a global health database storing epidemiological data. The vision test management platform can be configured to cross-reference the data collected from its user accounts with the epidemiological data to identify an emerging pattern and a public health concern. For example, a teenager's vision data may be collected and analyzed during an extended duration of time (e.g., 10 years) to identify an individual vision development trend and was cross-referenced with an average vision development trend extracted from the global health database. A doctor can rely on a cross-referencing result to determine whether the individual vision development trend is normal or whether the teenager's eyesight drops faster than average teenagers. As such, various embodiments of the vision test management platform may integrate biometric data and global health analytics and provides a secure, personalized, and interactive environment for vision testing, which can improve precision and user experience of vision assessments and contributes to broader public health monitoring and research initiatives.

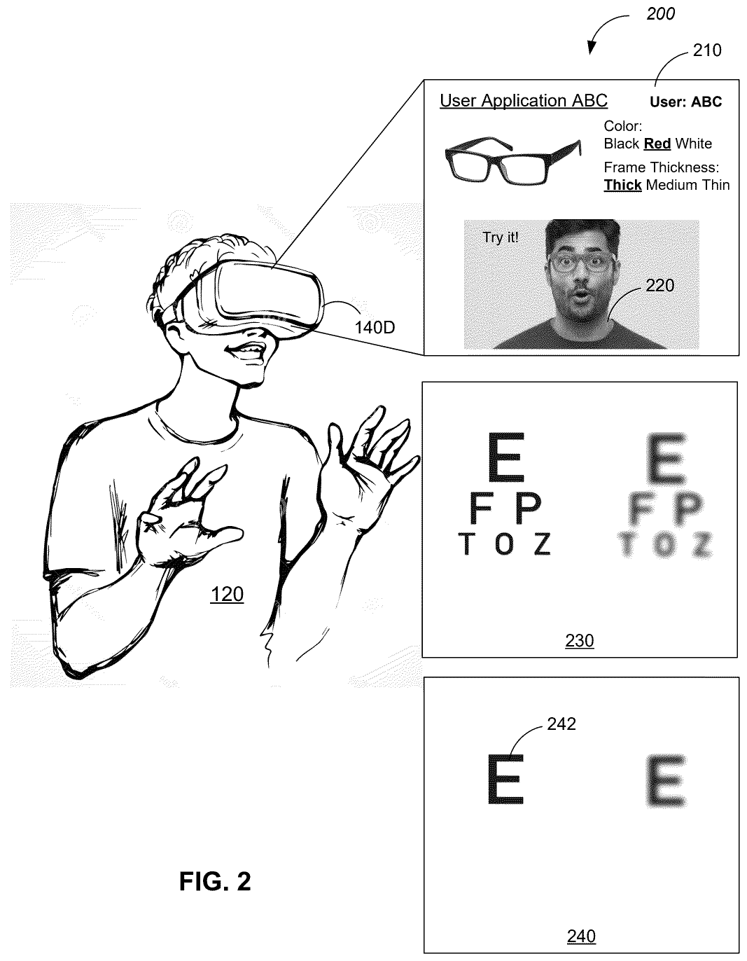

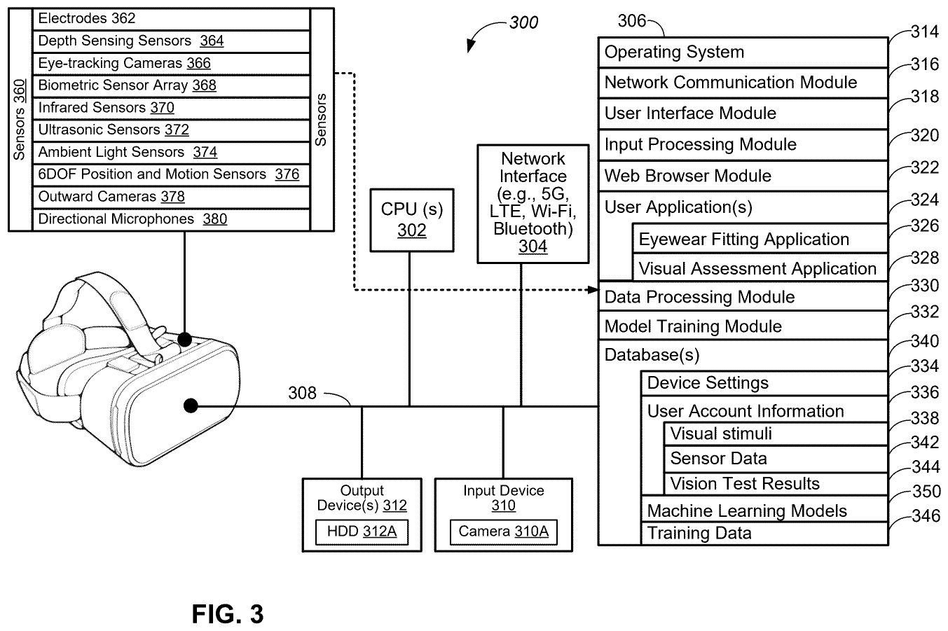

FIG. 2 is an environment 200 in which a computer device 140 (e.g., a headset device 140D) is applied to facilitate visual assessment or eyewear fitting, in accordance with some embodiments. The XR headset device 140D may be communicatively coupled within the data processing environment 100. The XR headset device 140D may include one or more cameras (e.g., a visible light camera, a depth camera), a microphone, a speaker, one or more inertial sensors (e.g., gyroscope, accelerometer), and a display. In some embodiments, the camera may capture hand gestures of a user wearing the XR headset device 140D. In some embodiments, the microphone may record ambient sound includes user's voice commands. The XR headset device 140D may execute a client-side eyewear fitting application 326 or a client-side visual assessment application 328 (FIG. 3) via a user account associated with a user 120 (e.g., an optometrist user, an optician user, a patient user). In some implementations, a computer device 140 (e.g., a mobile phone 140C) distinct from the XR headset device 140D can be used to implement the client-side eyewear fitting application 326 or visual assessment application 328 (FIG. 3).

In some embodiments, a first user interface 210 can be displayed on a computer device 140 (e.g., the headset device 140D) associated with the user 120. In some embodiments, an eyewear can be tried on or displayed as being worn by a 2D or 3D image 220 of the user 120. The server 102 or computer device 140 may receive, from the first user interface 210, a user feedback message indicating an issue, requesting further improvement, or confirming a fit. In some embodiments, a second user interface 230 can be displayed on a computer device 140 associated with the user 120. The second user interface 230 may include a plurality of optotypes (e.g., six optotypes E, F, P, T, O, and Z) having different sizes. In some embodiments, a third user interface 240 can be displayed on a computer device 140 associated with the user 120. The second user interface 230 can display a temporal sequence of optotypes having respective sizes. Each optotype of a corresponding size can be displayed at one time.

FIG. 3 is a block diagram of a computer system 300 (e.g., including a headset device 140D, a server, or a combination thereof) configured to implement vision assessment or eyewear fitting, in accordance with some embodiments. The computer system 300 can include one or more processing units (CPUs) 302, one or more network interfaces 304, memory 306, and one or more communication buses 308 for interconnecting these components (sometimes called a chipset). The computer system 300 may include one or more input devices 310 that facilitate user input, such as a keyboard, a mouse, a voice-command input unit or microphone, a touch screen display, a touch-sensitive input pad, a gesture capturing camera, or other input buttons or controls. Furthermore, in some embodiments, the computer device 140 of the computer system 300 may use a microphone for voice recognition or an eye tracking camera 366 for tracking eyeball movement. In some implementations, the computer device 140 may include one or more optical cameras (e.g., an RGB camera), scanners, or photo sensor units for capturing images. The computer system 300 may also include one or more output devices 312 that enable presentation of user interfaces 210 and media content. The one or more output devices 312 may include one or more speakers and/or one or more visual displays.

The computer system 300 may include one or more sensors 360, which further may include one or more of: a plurality of electrodes 362, one or more depth sensing sensors 364, one or more eye tracking cameras 366, a biometric sensor array 368, one or more infrared sensors 370, one or more ultrasonic sensors 372, one or more ambient sensors 374, one or more motion sensors (e.g., six degree of freedom (6DOF) position and motion sensors 376), one or more outward camera 378, and one or more directional microphones 380. It is noted that the one or more sensors 360 can also be included in the input device 310 and used to collect data to the computer system 300.

Memory 306 may include high-speed random-access memory, such as DRAM, SRAM, DDR RAM, or other random-access solid state memory devices; and, optionally, may include non-volatile memory, such as one or more magnetic disk storage devices, one or more optical disk storage devices, one or more flash memory devices, or one or more other non-volatile solid state storage devices. Memory 306, optionally, may include one or more storage devices remotely located from one or more processing units 302. Memory 306, or alternatively the non-volatile memory within memory 306, may include a non-transitory computer readable storage medium. In some implementations, memory 306, or the non-transitory computer readable storage medium of memory 306, may store the following programs, modules, and data structures, or a subset or superset thereof:
Operating system 314 including procedures for handling various basic system services and for performing hardware dependent tasks;
Network communication module 316 for connecting each server 102 or computer device 140 to other devices (e.g., server 102, computer device 140, or storage 106) via one or more network interfaces 304 (wired or wireless) and one or more communication networks 108, such as the Internet, other wide area networks, local area networks, metropolitan area networks, and so on;
User interface module 318 for enabling presentation of information (e.g., a graphical user interface for application(s) 324, widgets, websites and web pages thereof, and/or games, audio and/or video content, text, etc.) at each computer device 140 via one or more output devices 312 (e.g., displays, speakers, etc.);
Input processing module 320 for detecting one or more user inputs or interactions from one of the one or more input devices 310 and interpreting the detected input or interaction;
Web browser module 322 for navigating, requesting (e.g., via HTTP), and displaying websites and web pages thereof may include a web interface for logging into a user account associated with a computer device 140 or another electronic device, controlling the computer device if associated with the user account, and editing and reviewing settings and data that are associated with the user account;
One or more user applications 324 for execution by the computer system 300 (e.g., games, social network applications, smart home applications, extended reality application, and/or other web or non-web-based applications for controlling another electronic device and reviewing data captured by such devices), where in some embodiments, an eyewear fitting application 326 can be executed to implement eyewear fitting, and has a plurality of user accounts associated with a plurality of users 120 (e.g., technician users and eyewear users), and in some embodiments, a visual assessment application 328 can be executed to evaluate eyesight of a patient user, and has a plurality of user accounts associated with a plurality of users 120 (e.g., an optometrist user, a patient user);
Data processing module 330 for processing data associated with the user applications 324, e.g., using machine learning models 350;
Model training Module 332 for obtaining training data 346 and training machine learning models 350; and
One or more databases 340 for storing at least data including one or more of:
Device settings 334 including common device settings (e.g., service tier, device model, storage capacity, processing capabilities, communication capabilities, etc.) of the computer system 300;
User account information 336 for the one or more user applications 324, e.g., user names, security questions, account history data, user preferences, and predefined account settings, where in some embodiments, the user account information 336 may include facial measurements and one or more virtual fitting parameters associated with associated with a user account of an eye fitting application 326, and in some embodiments, the user account information 336 may include visual stimuli 338, sensor data 342, and vision test results 344 associated with a user account of a visual assessment application 328; and
Machine learning models 350 including parameters (e.g., weights, biases) used to implement vision test or select eyewear for eyewear users.

Each of the above identified elements may be stored in one or more of the previously mentioned memory devices, and corresponds to a set of instructions for performing a function described above. The above identified modules or programs (i.e., sets of instructions) need not be implemented as separate software programs, procedures, modules or data structures, and thus various subsets of these modules may be combined or otherwise re-arranged in some embodiments. In some embodiments, memory 306, optionally, stores a subset of the modules and data structures identified above. Furthermore, memory 306, optionally, stores additional modules and data structures not described above.

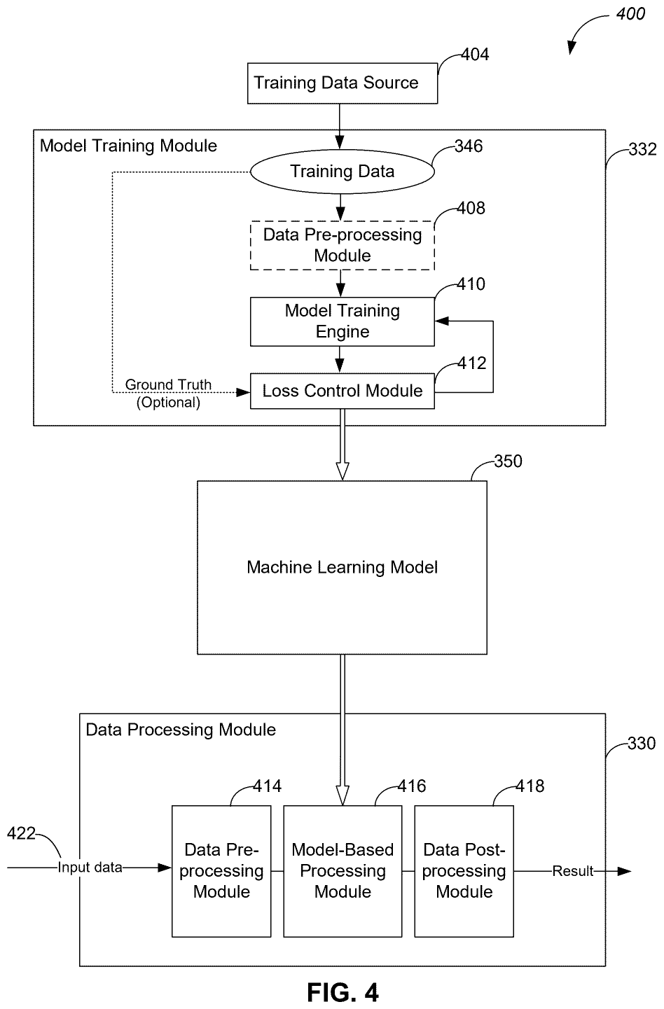

FIG. 4 is a block diagram of a machine learning system 400 for training and applying machine learning models 350 (e.g., for glass making), in accordance with some embodiments. The machine learning system 400 may include a model training module 332 establishing one or more machine learning models 350 and a data processing module 330 for processing input data 422 using the machine learning model 350. In some embodiments, both the model training module 332 and the data processing module 330 may be located within a computer device 140 (e.g., a VR headset), while a training data source 404 provides training data 346 to the computer device 140. In some embodiments, the training data source 404 may include the data obtained from the computer device 140 itself, from a server 102, from storage 106, or from another electronic device or computer device 140. Alternatively, in some embodiments, the model training module 332 may be located at a server 102, and the data processing module 330 may be located in a computer device 140. The server 102 can train the machine learning model 350 and provide the trained models 350 to the computer device 140 to process real-time input data 422 detected by the computer device 140. In some embodiments, the training data 346 provided by the training data source 404 may include a standard dataset widely used to train machine learning models 350. The input data 422 further may include sensor data. Further, in some embodiments, a subset of the training data 346 may be modified to augment the training data 346. The subset of modified training data may be used in place of or jointly with the subset of training data 346 to train the machine learning models 350.

In some embodiments, the model training module 332 may include a model training engine 410, and a loss control module 412. Each machine learning model 350 may be trained by the model training engine 410 to process corresponding input data 422 and implement a respective task. Specifically, the model training engine 410 may receive the training data 346 corresponding to a machine learning model 350 to be trained and process the training data to build the machine learning model 350. In some embodiments, during this process, the loss control module 412 can monitor a loss function comparing the output associated with the respective training data item to a ground truth of the respective training data item. In these embodiments, the model training engine 410 may modify the machine learning models 350 to reduce the loss, until the loss function satisfies a loss criterion (e.g., a comparison result of the loss function is minimized or reduced below a loss threshold). The machine learning models 350 may thereby be trained and provided to the data processing module 330 of a computer device 140 to process real-time input data 422 from the computer device 140.

In some embodiments, the model training module 402 may further include a data pre-processing module 408 configured to pre-process the training data 346 before the training data 346 is used by the model training engine 410 to train a machine learning model 350. For example, an image pre-processing module 408 is configured to format patients' eye images in the training data 346 into a predefined image format. For example, the preprocessing module 408 may normalize the images to a fixed size, resolution, or contrast level. In another example, an image pre-processing module 408 extracts a region of interest (ROI) corresponding to an eye area.

In some embodiments, the model training module 332 can use supervised learning in which the training data 346 may be labelled and include a desired output for each training data item (also called the ground truth, in some embodiments). In some embodiments, the desirable output may be labelled manually by people or automatically by the model training model 332 before training. In some embodiments, the model training module 332 may use unsupervised learning in which the training data 346 is not labelled. The model training module 332 is configured to identify previously undetected patterns in the training data 346 without pre-existing labels and with little or no human supervision. Additionally, in some embodiments, the model training module 332 may use partially supervised learning in which the training data is partially labelled.

In some embodiments, the data processing module 330 may include a data pre-processing module 414, a model-based processing module 416, and a data post-processing module 418. The data pre-processing modules 414 may pre-process input data 422 based on the type of the input data 422. In some embodiments, functions of the data pre-processing modules 414 are consistent with those of the pre-processing module 408. The data pre-processing modules 414 can convert the input data 422 into a predefined data format that is suitable for the inputs of the model-based processing module 416. The model-based processing module 416 may apply the trained machine learning model 350 provided by the model training module 332 to process the pre-processed input data 422. In some embodiments, the model-based processing module 416 can also monitor an error indicator to determine whether the input data 422 has been properly processed in the machine learning model 350. In some embodiments, the processed input data may be further processed by the data post-processing module 418 to create a preferred format or to provide additional information that can be derived from the processed input data. The data processing module 330 may use the processed input data to make eyewear glasses for a patient user.

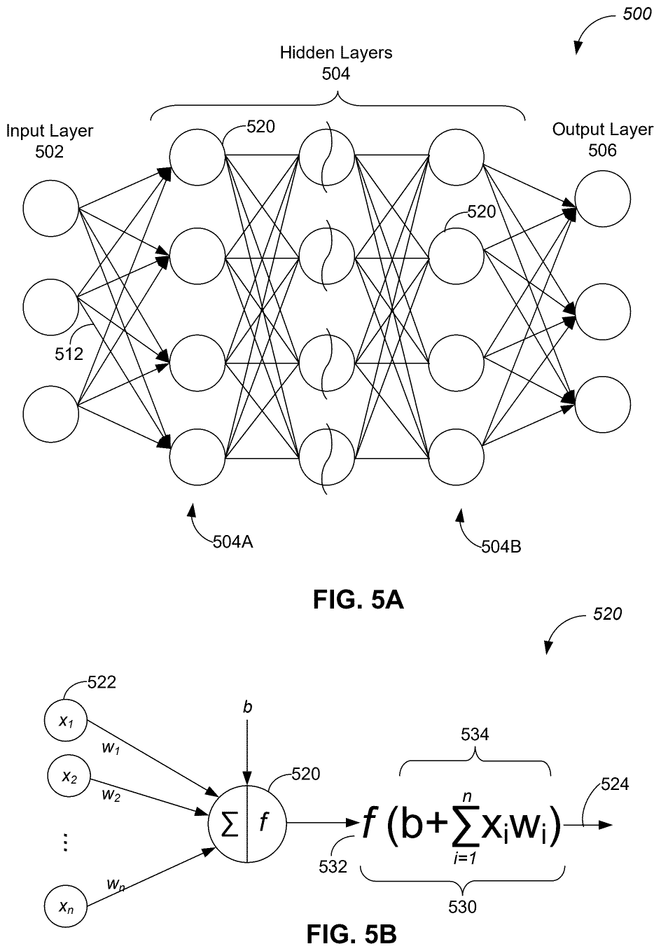

FIG. 5A is a structural diagram of an example neural network 500 applied to process input data in a machine learning model 350, in accordance with some embodiments. Further, FIG. 5B is an example node 520 in the neural network 500, in accordance with some embodiments. It should be noted that this description is used as an example only, and other types or configurations may be used to implement the embodiments described herein. The machine learning model 350 may be established based on the neural network 500. A corresponding model-based processing module 416 may apply the machine learning model 350 including the neural network 500 to process input data 422 that has been converted to a predefined data format. The neural network 500 may include a collection of nodes 520 that may be connected by links 512. Each node 520 may receive one or more node inputs 522 and applies a propagation function 530 to generate a node output 524 from the one or more node inputs. As the node output 524 is provided via one or more links 512 to one or more other nodes 520, a weight w associated with each link 512 may be applied to the node output 524. Likewise, the one or more node inputs 522 may be combined based on corresponding weights w1, w2, w3, and w4 according to the propagation function 530. In an example, the propagation function 530 is computed by applying a non-linear activation function 532 to a linear weighted combination 534 of the one or more node inputs 522.

The collection of nodes 520 may be organized into layers in the neural network 500. In general, the layers may include an input layer 502 for receiving inputs, an output layer 506 for providing outputs, and one or more hidden layers 504 (e.g., layers 504A and 504B) between the input layer 502 and the output layer 506. A deep neural network has more than one hidden layer 504 between the input layer 502 and the output layer 506. In the neural network 500, each layer may only be connected with its immediately preceding and/or immediately following layer. In some embodiments, a layer may be a “fully connected” layer because each node in the layer is connected to every node in its immediately following layer. In some embodiments, a hidden layer 504 may include two or more nodes that may be connected to the same node in its immediately following layer for down sampling or pooling the two or more nodes. In particular, max pooling may use a maximum value of the two or more nodes in the layer for generating the node of the immediately following layer.

In some embodiments, a convolutional neural network (CNN) may be applied in a machine learning model 350 to process input data. The CNN employs convolution operations and belongs to a class of deep neural networks. The hidden layers 504 of the CNN include convolutional layers. Each node in a convolutional layer may receive inputs from a receptive area associated with a previous layer (e.g., nine nodes). Each convolution layer may use a kernel to combine pixels in a respective area to generate outputs. For example, the kernel may be to a 3×3 matrix including weights applied to combine the pixels in the respective area surrounding each pixel. Video or image data can be pre-processed to a predefined video/image format corresponding to the inputs of the CNN. In some embodiments, the pre-processed video or image data may be abstracted by the CNN layers to form a respective feature map. In this way, video and image data can be processed by the CNN for video and image recognition or object detection.

In some embodiments, a recurrent neural network (RNN) is applied in the machine learning model 350 to process input data 422. Nodes in successive layers of the RNN follow a temporal sequence, such that the RNN exhibits a temporal dynamic behavior. In an example, each node 520 of the RNN has a time-varying real-valued activation. It is noted that in some embodiments, two or more types of input data may be processed by the data processing module 330, and two or more types of neural networks (e.g., both a CNN and an RNN) may be applied in the same machine learning model 350 to process the input data jointly.

The training process is a process for calibrating all of the weights wi for each layer of the neural network 500 using training data 346 that is provided in the input layer 502. The training process typically may include two steps, forward propagation and backward propagation, which may be repeated multiple times until a predefined convergence condition is satisfied. In the forward propagation, the set of weights for different layers may be applied to the input data and intermediate results from the previous layers. In the backward propagation, a margin of error of the output (e.g., a loss function) is measured (e.g., by a loss control module 412), and the weights may be adjusted accordingly to decrease the error. The activation function 532 can be linear, rectified linear, sigmoidal, hyperbolic tangent, or other types. In some embodiments, a network bias term b may be added to the sum of the weighted outputs 534 from the previous layer before the activation function 532 is applied. The network bias b may provide a perturbation that helps the neural network 500 avoid over fitting the training data. In some embodiments, the result of the training may include a network bias parameter b for each layer.

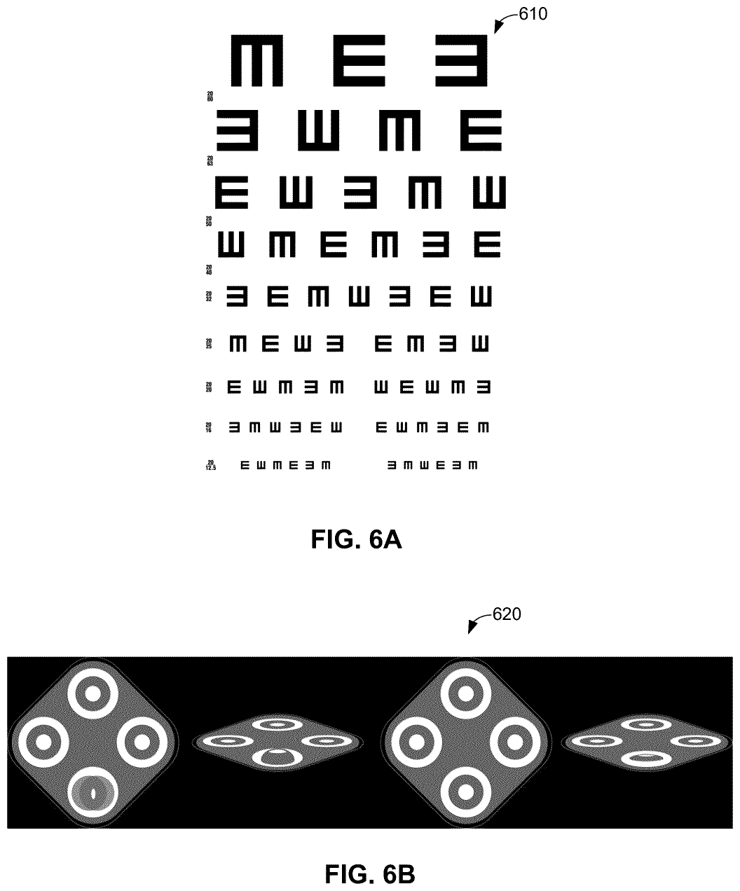

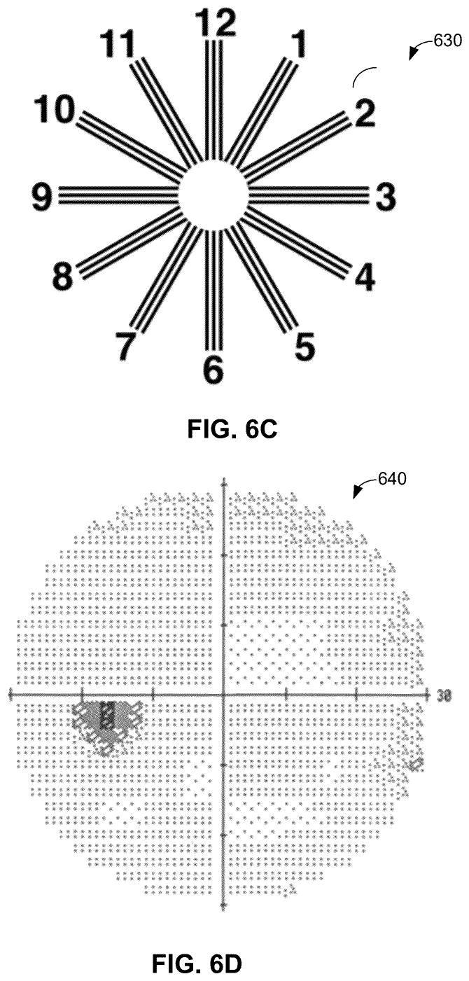

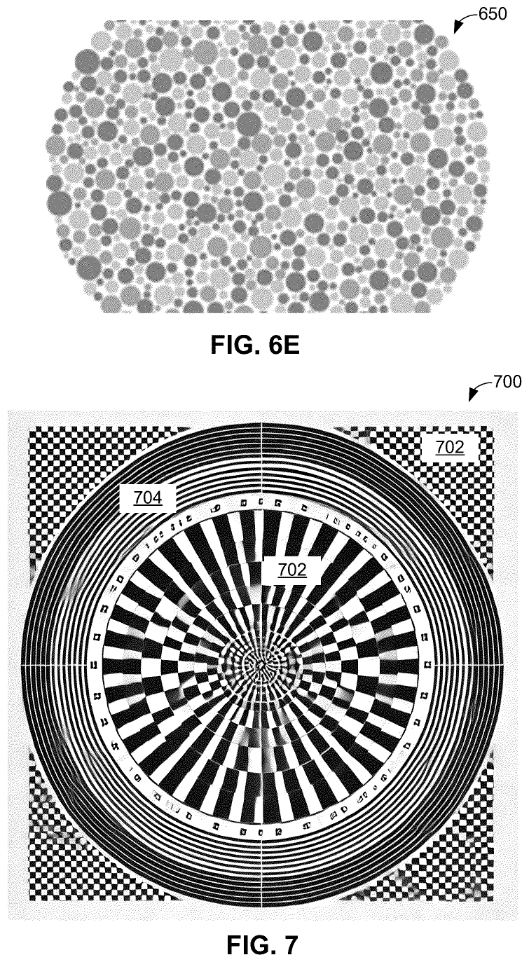

In some embodiments of the present disclosure, a vision test is implemented in a headset device 140D configured to display a user interface creating a three-dimensional (3D) virtual environment. Examples of a vision test implemented in the 3D virtual environment include, but are not limited to a visual acuity test, a visual field test, a visual depth test, a color blindness test, a retinoscopy, a test for stereopsis, a refraction test, an astigmatism test, and a contact lens exam. FIG. 6A is an example “tumbling E” chart 610 applied in a visual acuity test, in accordance with some embodiments. FIGS. 6B, 6C, 6D, and 6E are example patterns 620, 630, 640, and 650 applied in an astigmatism test, a stereopsis test, a visual field test, and a color blindness test, in accordance with some embodiments.

FIG. 7 is another example visual pattern 700 applied to test visual acuity and astigmatism, in accordance with some embodiments. The visual pattern 700 integrates a grid pattern 702 and concentric rings 704. The grid pattern 702 may include evenly spaced horizontal and vertical lines, creating a checkerboard pattern. The grid pattern 702 may be configured to identify distortions in straight lines, which can indicate issues with visual acuity and astigmatism. The concentric rings 704 may expand outward from a center of the visual pattern 700 and can assist in detecting radial distortions, which are common indicators of astigmatism. The visual pattern 700 may be depicted in high-contrast black and white, which ensures maximum clarity and reduces the potential for color-related distortions, making it easier to detect any visual impairment or defect.

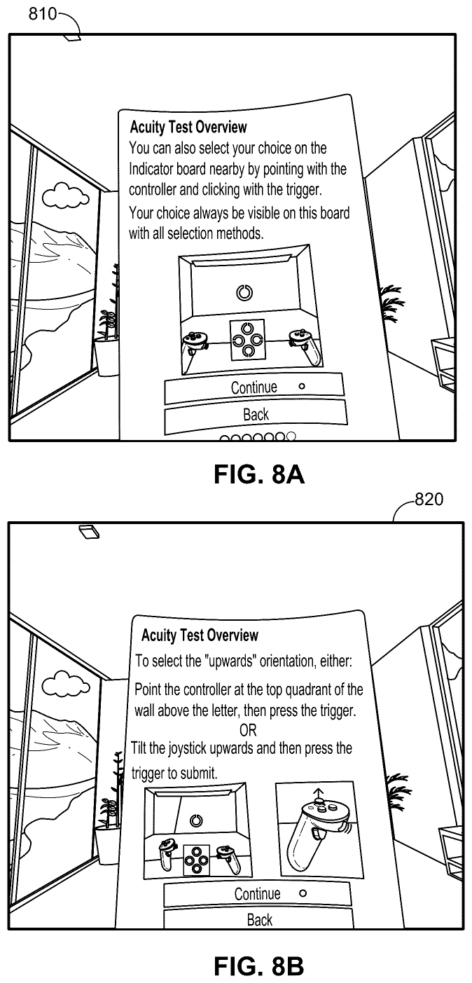

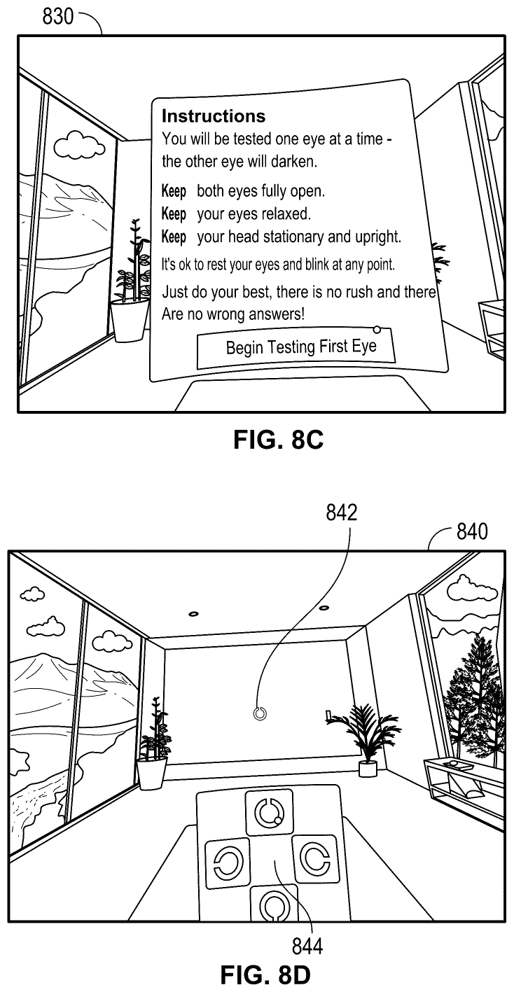

FIGS. 8A-8D include four diagrams of example graphical user interfaces 810, 820, 830, and 840 rendered to determine a visual acuity score in a virtual environment created by a headset device 140D, in accordance with some embodiments. The user interface 810 may display an information page including instructions on controlling a headset device 140D to select one of a plurality of optotype candidates to match a target optotype displayed in the virtual environment. The user interface 820 may display an information page including two optional ways of using the controller to select the one of the plurality of optotype candidates. The user interface 830 may display an information page including general guidelines on a visual acuity assessment process. The user interface 840 may display an optotype 842 that is projected on a screen that has a first distance L1 from a user's position in the virtual environment. In a second distance L2 near the user, a selection panel 844 including a plurality of optotype candidates may be displayed, prompting the user to select one of the optotype candidates that matches the optotype 842. In some embodiments, in response to a user selection of the one of the optotype candidates, the optotype 842 displayed in the first distance L1 may be updated with a new optotype 842. Further, in some embodiments, the new optotype 842 may spin at a fast rate for a shortened duration of time (e.g., 2 seconds), before it settles in place of the original optotype 842. In an example, the optotype 842 may spin and gradually shrink in size during the shortened duration of time.

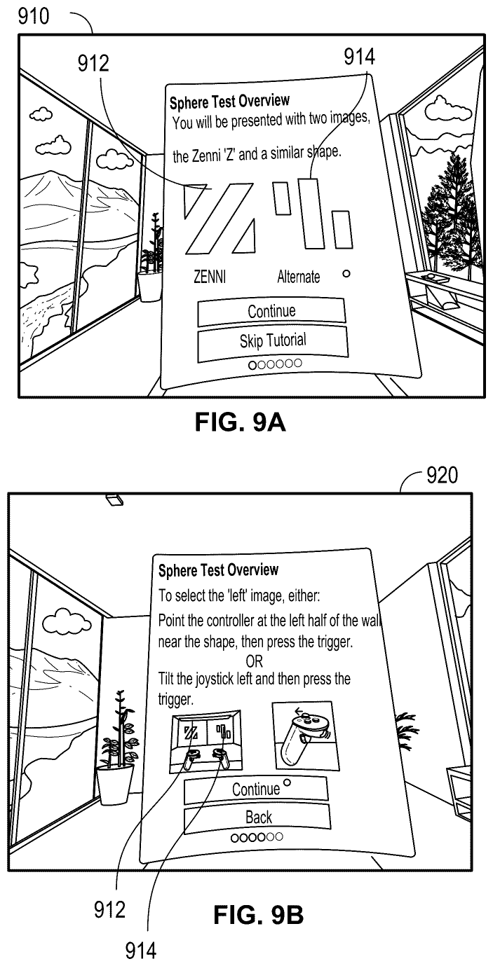

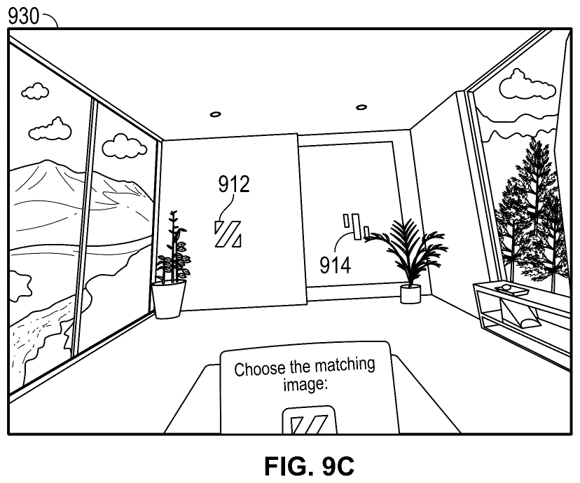

FIGS. 9A-9C include three diagrams of example graphical user interfaces 910, 920, and 930 rendered to determine a nearsighted or farsighted power in a virtual environment created by a headset device 140D, in accordance with some embodiments. The user interface 910 may display an information page explaining that two target optotypes 912 and 914 may be displayed in the virtual environment. The user interface 920 may display an information page including two optional ways of using the controller to select one of the two target optotypes 912 and 914. The user interface 930 may display two target optotypes 912 and 914 that may be projected on a screen that has a first distance L1 from a user's position in the virtual environment. In this example, the target optotype 912 located on the left is highlighted (e.g., by being displayed in a colored background). In a second distance L2 near the user, a confirmation panel 932 may be displayed, prompting the user to select one of the two target optotypes 912 and 914. In some embodiments, in response to a user selection of the one of the two target optotypes 912 and 914, the two target optotypes 912 and 914 displayed in the first distance L1 may be updated with a new pair of two target optotypes 912 and 914. Further, in some embodiments, each optotype 912 or 914 may spin at a fast rate for a shortened duration of time (e.g., 2 seconds), before it settles in place of the original optotype 912 or 914. In an example, the optotype 912 or 914 may spin and gradually shrink in size during the shortened duration of time.

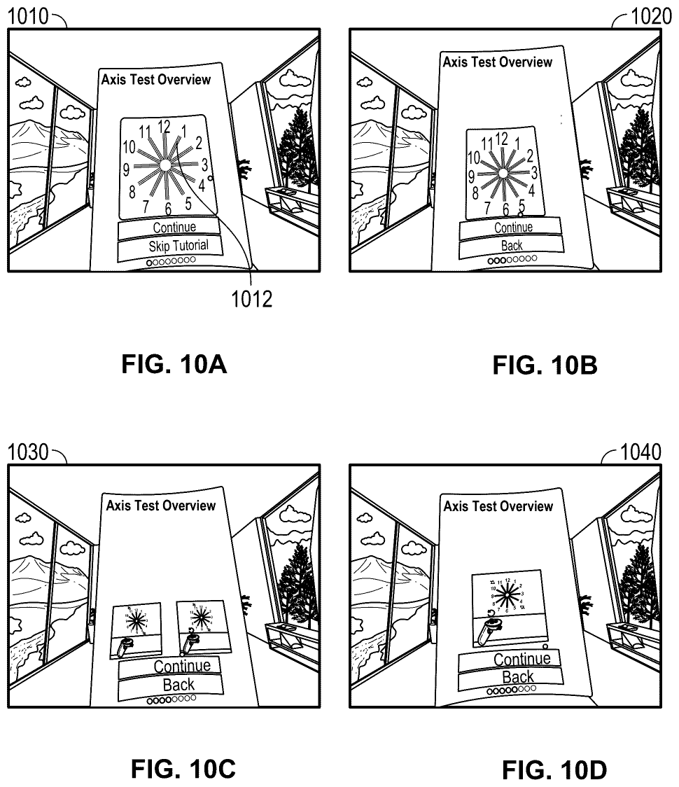

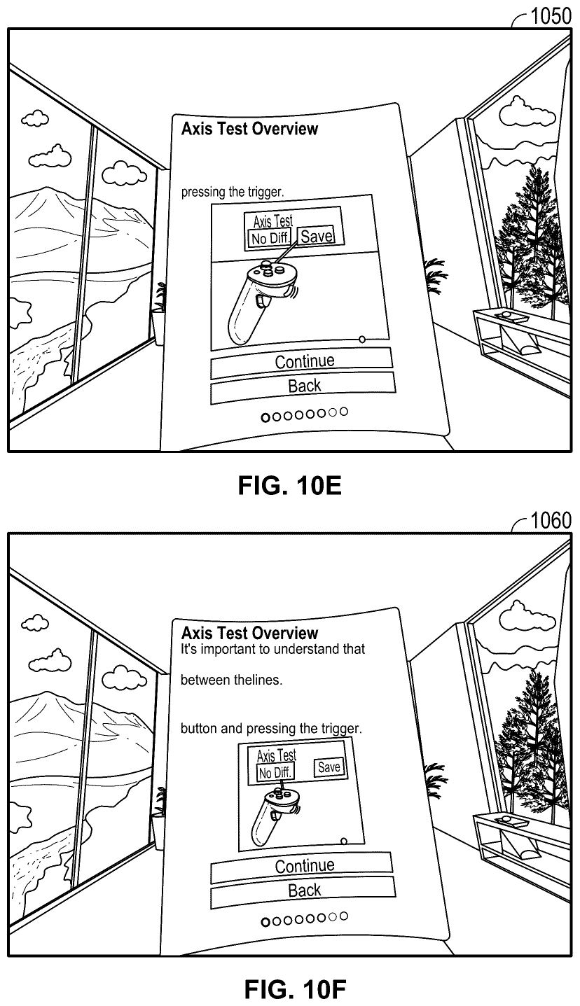

FIGS. 10A-10F include six diagrams of example graphical user interfaces 1010, 1020, 1030, 1040, 1050, and 1060 rendered to determine eye stigmatism in a virtual environment created by a headset device 140D, in accordance with some embodiments. The user interface 1010 may display an information page explaining that a clock diagram of converging numbered lines 1012 (which is a type of optotype) is displayed in the virtual environment. For example, the user interface 1010 may include a message, e.g., “You will be presented with a clock diagram of converging numbered lines.” The user interface 1020 may display an information page explaining what is selected on the clock diagram of converging numbered lines 1012 displayed in the virtual environment. For example, the user interface 1010 may include a message, e.g., “Your task is to identify if any of these sets of lines appear clearer, crisper, or darker than other.” The user interface 1030 may display an information page including two optional ways of using the controller to select lines on the clock diagram of converging numbered lines 1012. For example, the user interface 1010 may include a message, e.g., “Make a selection by either pointing the controller at the lines on the clock, then pressing the trigger” and “Rotating the joystick to move the indicator arrows around the clock.” The user interface 1040 may display an information page illustrating an embodiment having equally clear lines on the clock diagram of converging numbered lines 1012. For example, the user interface 1010 may include a message, e.g., “If two sets of neighboring lines seem to both stand out as equally clear, you can move the indicator arrows to a halfway point between those lines.”

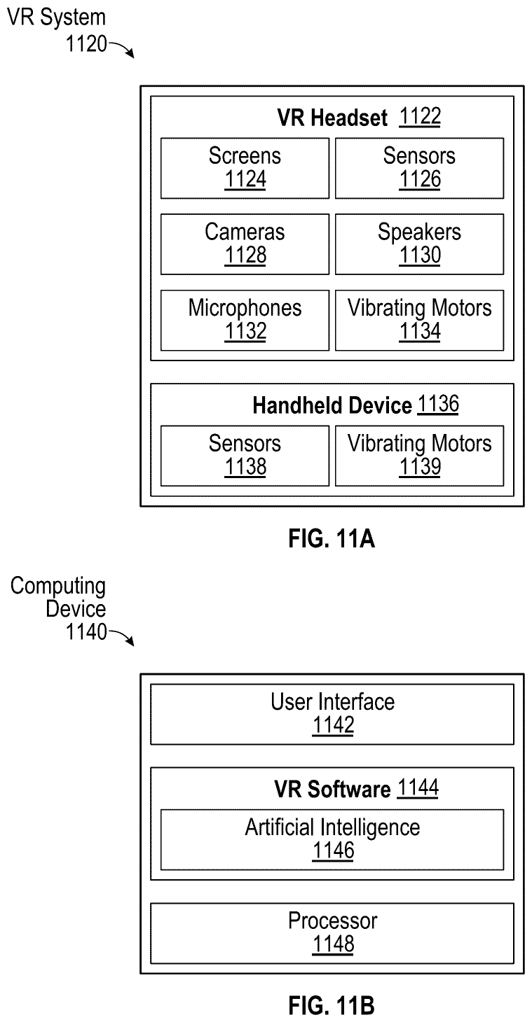

Referring to FIG. 10E, the user interface 1050 may display an information page including an instruction using the controller to submit a selection. For example, the user interface 1010 may include a message, e.g., “After selecting a set of lines, submit your choice with the ‘Done’ button below by pointing to the controller at the button and pressing the trigger.” Further, referring to FIG. 10F, the user interface 1060 may display an information page including an instruction using the controller to indicate that no difference is observed on the clock diagram of converging numbered lines 1012. For example, the user interface 1010 may include a message, e.g., “It's important to understand that not everybody will see a difference between the lines” and “In this case, simply select ‘No Difference’ below, by positioning the controller at the button and pressing the trigger.” FIGS. 11A and 11B illustrate the components of the vision evaluation system: the VR system and the computing device, in accordance with some embodiments. The VR system 1120 is one of the computer devices 140 described with respect to FIG. 1 (e.g., the headset device 140D). The VR system 1120 may include a VR headset 1122 and/or a handheld device 1136. The VR headset 1122 is worn by a patient and includes screens 1124 for displaying a controlled environment (e.g., visual anomalies, visual tests/exams, visual tasks/exercises, etc.) for the patient. The VR headset 1122 also includes sensors 1126 (e.g., eye-tracking sensors) for receiving the patient's responses to the controlled environment. The sensors 1126 can be used to measure data such as the patient's blink rate, pupil diameter, heart rate, skin conductance, etc. The sensors 1126 may be constructed and operable in accordance with any variety of conventional technologies.

In some embodiments, the VR headset 1122 also includes cameras 1128 and/or microphones 1132 for receiving the patient's responses to the controlled environment. For example, the microphones 1132 can be used to receive a patient's verbal responses to the controlled environment. Similarly, the cameras 1128 of the VR headset 1122 are configured to collect additional data about and input from the patient—in particular, visual inputs from the patient such as blink rate, pupil dilation, saccades, smooth pursuit movements, vergence movements, and vestsibulo-ocular movements.

In other embodiments, the VR headset 1122 also includes speakers 1130 and/or vibrating motors 1134 for providing audio and tactile feedback and/or stimuli to the patient. For example, the vibrating motors 1134 can output haptic responses based on the patient's responses to the controlled environment.

The handheld device 1136 is held by the patient and is in electronic communication with the VR headset 1122. The handheld device 1136 includes sensors 1138 for receiving the patient's responses to the controlled environment. The sensors 1138 can measure the patient's grip strength, heart rate, skin conductance, etc. The sensors 1138 can also receive input from the patient (e.g., the patient presses a button on the handheld device 1136 to confirm that they are perceiving a visual anomaly displayed on the screens 1124). Optionally, the handheld device 1136 also includes vibrating motors 1139. The vibrating motors 1139 can provide tactile feedback and/or stimuli to the patient based on the patient's responses to the controlled environment. For example, if the patient is incorrectly responding to a visual test, the vibrating motors 1139 of the handheld device 1136 can provide a haptic response to the patient to indicate that the patient's response is incorrect.

The components of the computing device 1140 are shown in FIG. 11B. The computing device 1140 is one of the computer devices 140 described with respect to FIG. 1 (e.g., the desktop computers 140A, tablet computers 140B, or mobile phones 140C). The computing device 1140 includes a user interface 1142, VR software 1144, and a processor 1148. The user interface 1142 can display the patient's responses (e.g., voluntary responses such as test answers or feedback and involuntary responses such as biological reactions) to the controlled environment. These responses can be accessed at the user interface 1142 by the patient, a physician, a parent (e.g., where the patient is a child), etc. The user interface 1142 can also receive input from the patient, the physician, or the parent. For example, the physician can indicate which tests the patient must undergo, input details about the patient's medical history, etc. Similarly, the parent can indicate what areas of optical health they want their child to focus on or provide information about their child's behavior or problem areas.

The VR software 1144 of the computing device 1140 includes the artificial intelligence 1146. Optionally, the VR software 1144 includes a machine-learning algorithm, an adaptation algorithm, or a multi-layered algorithm.

The processor 1148 of the computing device 1140 implements the VR software 1144 and evaluates the patient's responses to the controlled environment.

### Simulating Visual Anomalies

Some implementations of the vision evaluation methods simulate visual anomalies (or visual disturbances) to help diagnose ocular conditions (e.g., cataracts, glaucoma, macular degeneration, retinal detachment, etc.). These methods can be implemented using the vision evaluation system described with respect to FIGS. 11A-11B, which makes these methods far less invasive than traditional vision evaluation methods.

In some embodiments, the VR headset 1122 is in electronic communication with the computing device 1140. The computing device 1140 is configured to process the data or input collected by the sensors 1126 and use that data or input to control an environment displayed on the screens 1124. Controlling the environment displayed on the screens 1124 includes causing visual anomalies to be displayed on the screens 1124 and changing those visual anomalies based on the data or input. The computing device 1140 continues to change the visual anomalies displayed on the screens 1124 until the patient indicates that the visual anomalies displayed are of the same intensity and nature of the visual anomalies that the patient experiences in real life.

By understanding the visual anomalies that the patient experiences in real life, the vision evaluation method described herein can evaluate the patient for an ocular condition more quickly and more accurately, as compared to a standard physician conversing with the patient to conduct the evaluation. This method increases the accuracy of a diagnosis by at least 50%. This is because the vision evaluation method not only facilitates the faster collection data and input (as compared to a standard physician), but also because this vision evaluation method allows the physician (or an artificial intelligence) leads to a more precise understanding of the visual anomalies experienced by the patient.

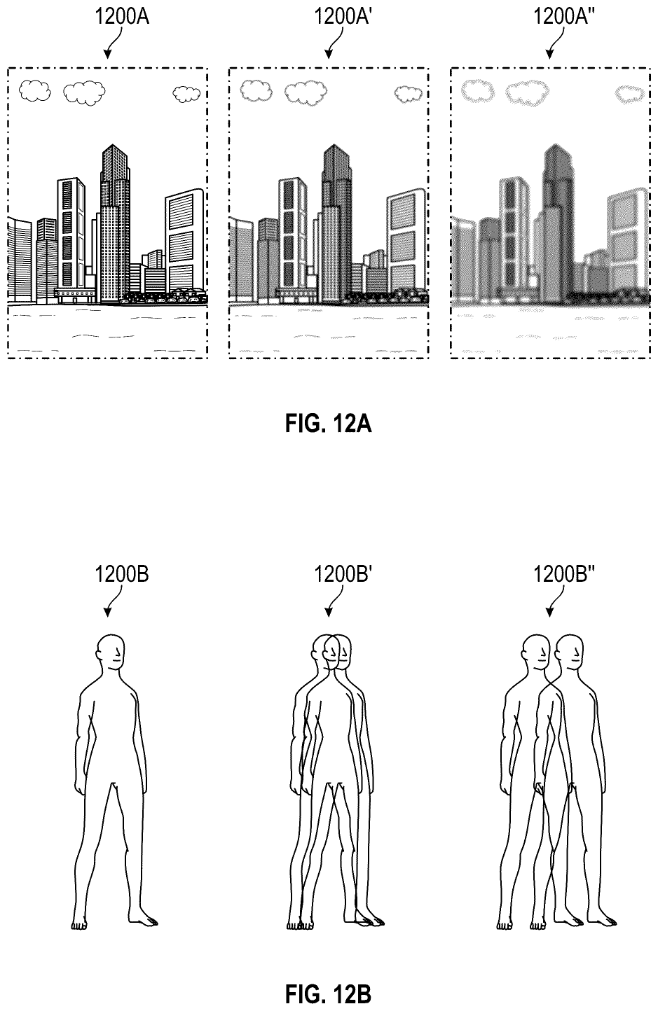

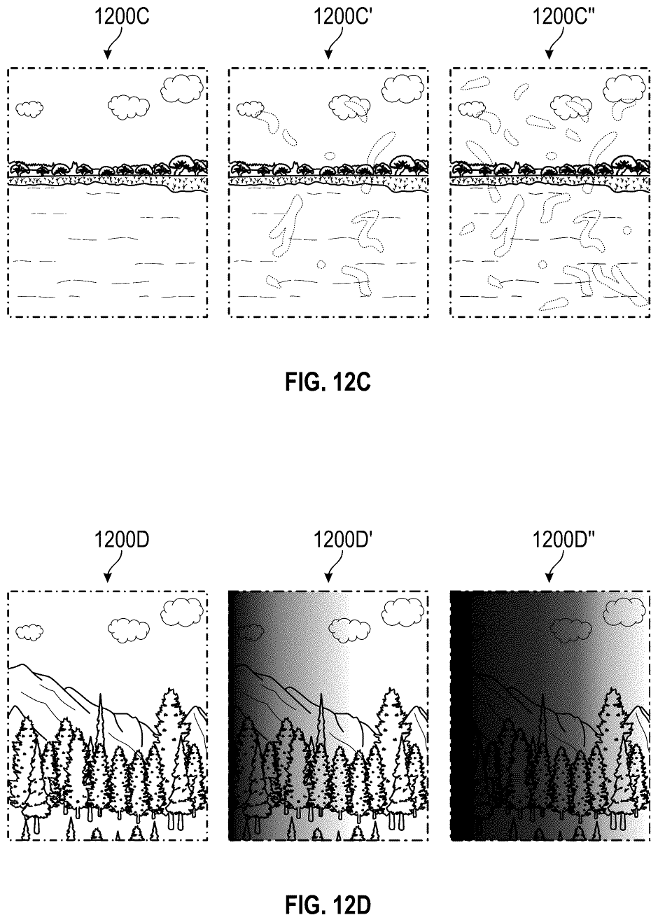

The patient starts by donning the VR headset 1122, and the sensors 1126 on the VR headset 1122 start collecting data about the patient. The data can include biodata such as scarring, skin tone around the patient's eyes, blink rate, pupil diameter, etc. The data can also include the patient's previous prescriptions and ocular history (e.g., interior segment, potential disease like cataracts, corneal abrasions, scarring, issues with posterior segment, retinopathy, retinal scarring, etc.), which can be inputted into the computing device 1140. The computing device 1140 processes this data in order to develop the features (e.g., visual anomalies, brightness, contrast, etc.) of a first environment displayed on the screens 1124 of the VR headset 1122. For example, for a patient with corneal abrasions, the virtual environment might include features such as blurred vision, light sensitivity, and glare. Corneal abrasions can cause the surface of the eye to become uneven, leading to scattered light entering the eye. The VR system would simulate this effect by creating a halo effect around light sources, increased glare from surfaces, and overall blurred vision, especially when looking at bright objects or during tasks requiring fine detail. The environment might also have adjusted brightness levels to simulate the discomfort that bright light causes to someone with corneal abrasions. The contrast might be reduced to simulate the difficulty in distinguishing objects in environments with varying lighting conditions. In another example, for a patient with retinopathy, which often results in damage to the retina, the VR system would introduce features such as dark spots (scotomas), reduced peripheral vision, and possibly fluctuating vision clarity. The virtual environment might include areas where the vision is obscured by dark spots or where peripheral vision is significantly reduced, making it difficult for the patient to navigate through the environment or detect objects outside his direct line of sight. For a patient with retinopathy, the brightness might be more evenly distributed (as compared to a patient with corneal abrasions) but with certain areas of the visual field being dimmer or appearing as shadowed, reflecting the damage to specific retinal areas. Contrast might be selectively reduced in the peripheral vision zones while keeping the central vision relatively clearer, mimicking the vision challenges faced by someone with retinopathy. The various environments and visual anomalies that the computing device 1140 can cause to be displayed on the screens 1124 are also described below with respect to FIGS. 12A-12D.

FIGS. 12A-12D illustrate examples of different visual anomalies with varying degrees of intensity being displayed in the environment on the screen of the VR headset, in accordance with some embodiments. FIG. 12A shows an environment 1200A that is sharp and has no visual anomaly, an environment 1200A′ that is mildly blurry, and an environment 1200A″ that is very blurry. The environments 1200A, 1200A′, and 1200A″ all demonstrate the same nature of visual anomaly (blur) at varying intensities (i.e., nonexistent intensity to high intensity). Another type of visual anomaly is diplopia (also referred to as double vision). FIG. 12B shows an environment 1200B with a person, an environment 1200B′ with the person being doubled and mostly overlapping, and an environment 1200B″ with the person being doubled and barely overlapping. Each environment 1200B, 1200B′, and 1200B″ demonstrate a different degree of diplopia. Similarly, FIG. 12C shows environments 1200C, 1200C′, and 1200C″, which each have varying amounts of floaters. Finally, FIG. 12D shows environments 1200D, 1200D′, and 1200D″, where the amount of landscape that is visible in the environment decreases from 1200D to 1200D″ to simulate varying intensities of field loss. Each of the environments illustrated in FIGS. 12A-12D can be displayed on the screens 1124 of the VR headset 1122.

As discussed above, the computing device 1140 controls a first environment displayed on the screens 1124 of the VR headset 1122 based on data collected about the patient. The first environment depicts a first visual anomaly, and the first visual anomaly can include one or more visual anomalies, including the visual anomalies described above with respect to FIGS. 12A-12D. In some embodiments, the first visual anomaly includes multiple types of visual anomaly displayed simultaneously in the first environment. For example, the environment can include the blur shown in environment 1200A′ and the field loss shown in environment 1200D′.

The patient perceives the first environment and provides a response in the form of a first input. The first input can include one or more of a patient's reaction time, eye movements, blink rate, or pupil dilation. This first input is received by the sensors 1126. Additionally, the first input can include verbal input from the patient (e.g., the patient can confirm or deny that the first environment accurately depicts what the patient perceives in real life). This verbal input can be received at microphones 1132 that are positioned on the VR headset 1122.

The first input is collected at the VR headset 1122 (i.e., by the sensors 1126 or the microphones 1132), such that the computing device 1140 can process the first input. Based on the first input, the computing device 1140 causes a second environment to be displayed on the screens 1124 of the VR headset 1122. The second environment depicts a second visual anomaly, where the second visual anomaly can have a different nature, frequency, and/or intensity than the first anomaly. For example, the second anomaly can include a more intense blur (e.g., the blur shown in environment 1200A″) and a more intense field loss (e.g., the blur shown in environment 1200D″) as compared to the first anomaly. In another example, the second anomaly has less intense blur than the first anomaly (e.g., the environment 1200A), no change to the field loss, and the addition of floaters (e.g., the environment 1200C′).

The patient next perceives the second environment and provides a response in the form of a second input. The sensors 1126 collect the second input, so that the computing device 1140 can process the second input. Depending on the characteristics of the second input, the computing device 1140 can continue displaying the second environment or cause a third environment to be displayed. For example, if the second input indicates that the second environment is an accurate depiction of what the patient perceives outside of the VR environment, then the computing device 1140 will continue to display the second environment and collect data and input from the patient. Alternatively, if the second input indicates that the second environment is not an accurate depiction, then the computing device 1140 will display a third, fourth, or fifth environment and continue collecting input from the patient until the environment is an accurate depiction of what the patient perceives outside of the VR environment.

Optionally, the computing device 1140 implements a machine-learning algorithm, and the machine-learning algorithm processes the input collected from the patient in response to the changing environments and determines the intensity, frequency, and nature of the visual anomalies displayed in the next environment. For instance, the machine-learning algorithm processes the first input and determines that the second anomaly should include a more intense blur and a more intense field loss. The machine-learning algorithm can also account for biodata, medical history, etc. while processing the input.

The computing device 1140 and the machine-learning algorithm facilitate real-time processing and analysis of the input from the patient, which allows for continuous and precise adaptation of the visual anomalies displayed in the environment. This method leads to a highly accurate diagnosis in a shorter period of time as compared to traditional vision evaluation methods. Specifically, this method can accomplish an evaluation that a standard physician conducts over two or more appointments in a single sitting. Moreover, because this method makes it possible to evaluate patients accurately and comprehensively in a single sitting instead of over multiple appointments, this method also reduces the cost of optical healthcare for patients.

In some embodiments, the computing device 1140 produces a report of the input collected from the patient in response to the changing environments. A physician reads and analyzes the report in order to determine the intensity, frequency, and nature of the visual anomalies displayed in the next environment. The physician controls the environment displayed on the screens 1124 of the VR headset 1122 through a web-based portal that is connected to the VR headset 1122 and the computing device 1140.

In other embodiments, the physician controls the environment displayed on the screens 1124 of the VR headset 1122 and communicates with the patient to determine whether the environment is an accurate depiction of what the patient sees outside of the VR environment. For example, the physician shows environment 1200A to the patient. However, the patient states that he typically sees more blur, so the physician increases the intensity of the blur to show environment 1200A″ to the patient. The patient states that this is too much blur, so the physician decreases the intensity of the blur to show environment 1200A′ to the patient. The physician and the patient continue this procedure until the patient confirms that the environment on the screens 1124 is an accurate depiction of what the patient typically sees outside of the VR environment. Through this method, the physician obtains a very accurate and objective understanding of what the patient perceives, which enables the physician to diagnose the patient's ocular condition more accurately.

The computing device 1140 uses the data and the input (which can include every set of input, the last few sets of input, or the last set of input) to evaluate the patient for an ocular condition. In particular, the computing device 1140 compares the data and the input with a database to evaluate the patient for one or more ocular conditions. The database comprises data and input from a control group of patients (i.e., patients with known visual health statuses).

Optionally, the patient is also evaluated to determine the severity and/or urgency of one or more ocular conditions.

In some embodiments, the computing device 1140 implements a machine-learning algorithm, and the machine-learning algorithm processes the data and the input to evaluate the patient for one or more ocular conditions. The machine-learning algorithm compares the data and the input with the database described above.

In other embodiments, the computing device 1140 produces a report of the input collected from the patient in response to the changing environments. A physician reads the report and uses the information obtained from the report to evaluate the patient for an ocular condition.

### Dynamically Conducting Visual Tests

Some implementations of the vision evaluation methods comprise dynamically conducting visual tests to evaluate a patient's visual health and assess various visual functions such as visual acuity, field of vision, contrast sensitivity, and color perception. These methods can be implemented using the vision evaluation system described with respect to FIGS. 11A-11B, which makes these methods far less invasive than traditional vision evaluation methods.

The VR system 1120 is in electronic communication with the computing device 1140. The computing device 1140 is configured to process the data or input collected by the sensors 1126 and use that data or input to control an environment displayed on the screens 1124. Controlling the environment displayed on the screens 1124 includes displaying ocular exams on the screens 1124 and dynamically changing the ocular exams being displayed based on the patient's responses to the ocular exams. The computing device 1140 continues to change the ocular exam displayed on the screens 1124 until the patient has been evaluated for all relevant ocular conditions (e.g., diabetic retinopathy, glaucoma, macular degeneration, refractive errors, etc.).

By dynamically changing the ocular exams being displayed, the vision evaluation method described herein can evaluate the patient for a wide variety of ocular conditions quickly and accurately. This method reduces the amount of time spent on any given ocular exam because it does not require the patient to complete each ocular exam that is traditionally necessary to provide a comprehensive evaluation. This method conducts a comprehensive evaluation at least two times faster than a standard clinician with five years of experience administering the ocular exams required to conduct a similar evaluation. This is accomplished by obviating the need for the patient to reach the end of any given ocular exam by collecting the patient's responses to the ocular exam in real-time, analyzing those responses, and utilizing those response to transition into a subsequent ocular exam. The difficulty and nature of the subsequent ocular exam is based on the patient's responses to the initial ocular exam. For instance, if a patient does not show symptoms for the initial ocular exam, the initial ocular exam is quickly ended in exchange for a subsequent ocular exam. Not only does this method enable the patient to take a multitude of ocular exams in a shortened period of time (which also reduces the patient's medical bills by reducing the number of appointments required for a complete evaluation), but this method also leads to a narrower and more nuanced ocular diagnosis because it facilitates a more comprehensive evaluation.

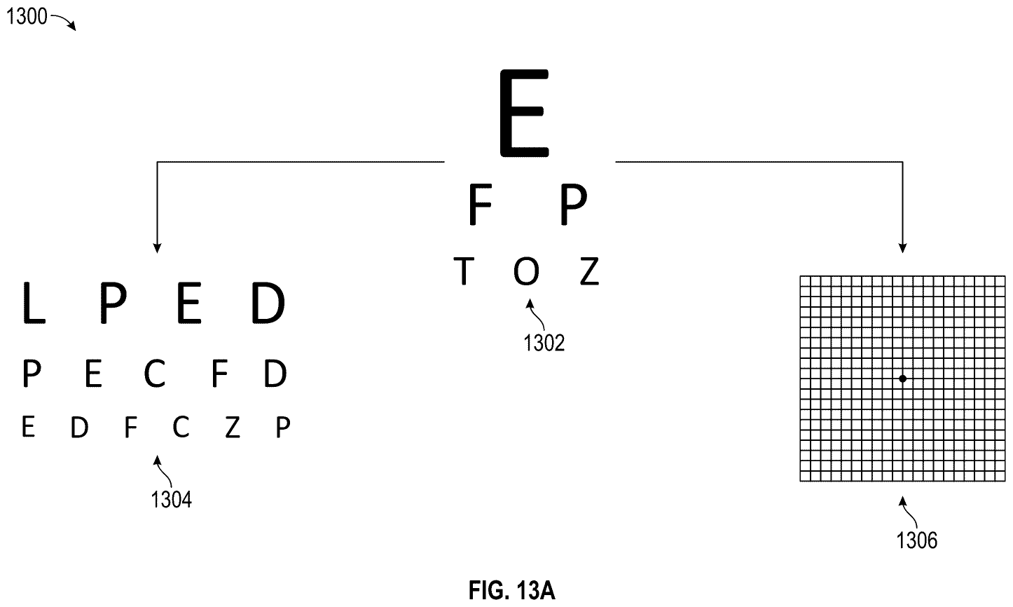

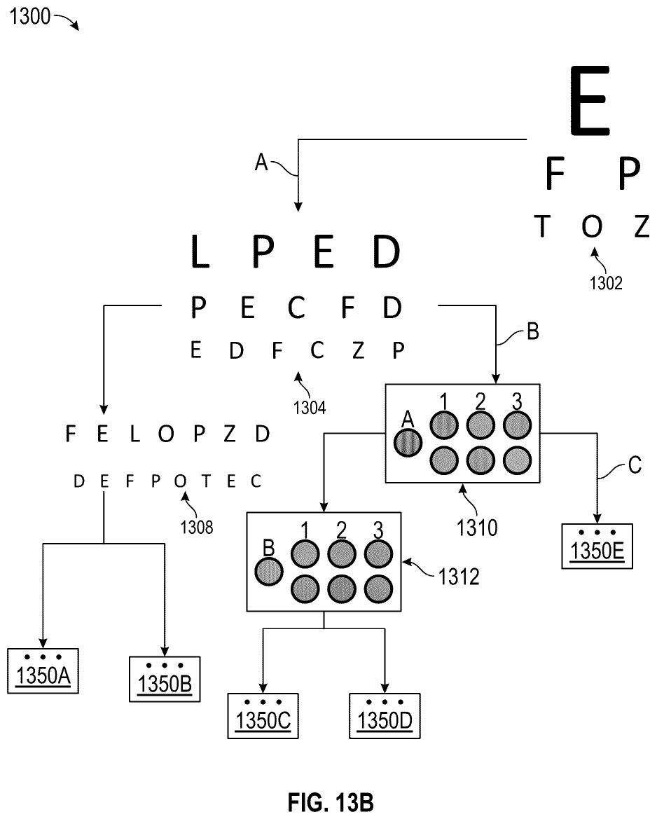

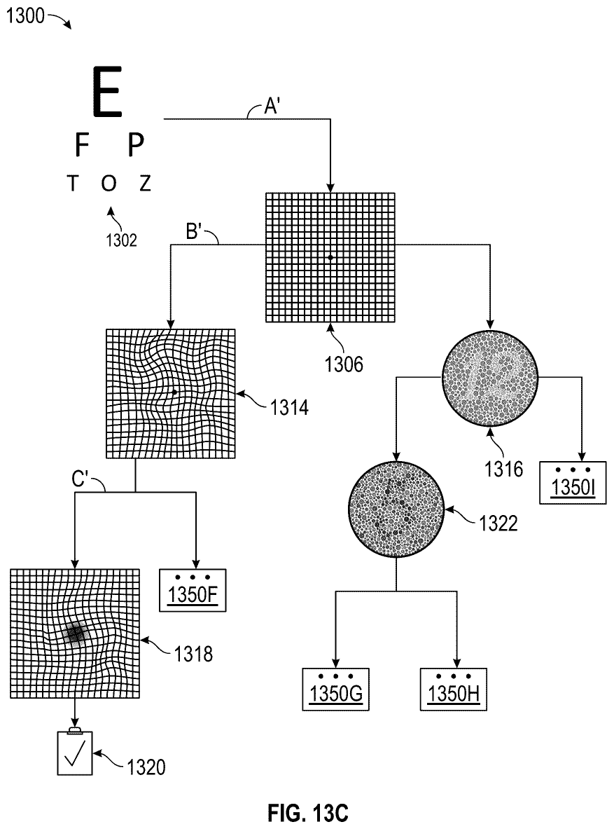

The process starts when the patient dons the VR headset 1122. Next, the computing device 1140 causes a first ocular exam to be administered on the screens 1124 of the VR headset 1122 to evaluate the patient for a first ocular condition. The types of ocular exam that can be administered on the screens 1124 are described in greater detail below with respect to FIGS. 13A-13C. In some embodiments, the computing device 1140 receives data about the patient, such as the patient's medical history and biodata. Optionally, the type of ocular exam conducted is influenced by the medical history and biodata of the patient.

As the patient responds to the first ocular exam, the VR headset 1122 receives a first input from the patient. The first input comprises both voluntary and involuntary responses from the patient. Moreover, the first input comprises audio, visual, and tactile inputs from the patient (e.g., verbal confirmations, blink rates, and button pushing). The computing device 1140 processes the first input and compares the first input with a database. The database can include data and input from a group of individuals who are known to have the first ocular condition.

Based on this comparison, the computing device 1140 decides whether to continue the first ocular exam in order to collect more information and evaluate the patient for the first ocular condition or to stop the first ocular exam and conduct a different exam. The computing device 1140 is processing the first input in real-time, so it is possible for the computing device 1140 to gather enough information to decide to stop the first ocular exam before the patient completes the first ocular exam. If the computing device 1140 decides to stop the first ocular exam, the computing device 1140 causes either a first ocular sub-exam or a second ocular exam to be displayed on the screens 1124.

The first ocular sub-exam is administered to evaluate the patient for a sub-condition of the first ocular condition (hereinafter referred to as a first ocular sub-condition). In some embodiments, the first ocular sub-condition is a symptom of the first ocular condition. As the patient responds to the first ocular sub-exam, the VR headset 1122 receives a first sub-input from the patient. The computing device 1140 processes the first sub-input and compares the first sub-input with a database. The database can include input from a group of individuals who are known to have the first ocular sub-condition.

The second ocular exam is administered to evaluate the patient for a second ocular condition. As the patient responds to the second ocular exam, the VR headset 1122 receives a second input from the patient. The computing device 1140 processes the second input and compares the second input with a database. The database can include input from a group of individuals who are known to have the second ocular condition.

FIGS. 13A-13D illustrate an example of the method of dynamically administering vision tests, in accordance with some embodiments. FIG. 13A displays the root node and the first internal nodes of a decision tree 1300, where the root node is a visual acuity test 1302, and the first internal nodes are a visual acuity sub-test 1304 and a field of vision test 1306. In this example, the visual acuity test 1302 is equivalent to the first ocular exam, the visual acuity sub-test 1304 is equivalent to the first ocular sub-exam, and the field of vision test 1306 is equivalent to the second ocular exam. The method starts by administering the visual acuity test 1302 through the VR headset 1122 and receiving the patient's responses to the visual acuity test 1302. Depending on how the patient's responses compare to the database of responses from individuals known to have visual acuity issues, the computing device 1140 will either continue administering the visual acuity test 1302 to collect more information, move onto the visual acuity sub-test 1304, or move onto the field of vision test 1306.

FIG. 13B displays the portion of decision tree 1300 that follows a path A-B-C. In this example, the computing device 1140 determined that the patient needed additional visual acuity testing based on the patient's responses to the visual acuity test 1302. As a result, the computing device 1140 followed path A and administered the visual acuity sub-test 1304. Based on the patient's responses to the visual acuity sub-test 1304, the computing device 1140 might have noticed that the patient demonstrated some issues with contrast sensitivity. In response, the computing device 1140 followed path B and administered the contrast test 1310. The patient's responses to the contrast test 1310 led the computing device 1140 to conclude that the patient did not have issues with contrast sensitivity, so the computing device 1140 followed path C to administer a different type of vision testb 1350E. The computing device 1140 continues this process until it completes the evaluation by cycling through all relevant vision tests or reaching a diagnosis.

FIG. 13C displays the portion of decision tree 1300 that follows a path A′-B′-C′ and leads to a diagnosis 1320. In this example, the computing device 1140 processed the patient's responses to the visual acuity test 1301 and determined that the patient did not require any additional visual acuity testing. In response to this determination, the computing device 1140 followed path A′ and administered field of vision test 1306. The patient's responses to the field of vision test 1306 might have indicated that the patient has a limited field of vision. In that case, the computing device 1140 used this information and followed path B′ to field of vision sub-test 1314. If the patient's responses to the field of vision sub-test 1314 indicated that the patient has a severely limited field of vision, then the computing device 1140 might follow path C′ to yet another field of vision sub-test 1318. This line of testing led to a diagnosis 1320.

In some embodiments, the computing device 1140 uses input collected from multiple lines of testing to develop the diagnosis. For example, throughout the testing process, the patient might undergo tests in the A-B-C path as well as the A′-B′-C′ path. This information can be combined to develop a very comprehensive and nuanced diagnosis of one or more ocular conditions and/or sub-conditions.

The computing device 1140 may include a user interface 1142 with a user interface progress tracker. The progress tracker can be a horizontal bar that fills from left to right (or vice versa), a vertical bar that fills from bottom to top (or vice versa), or a radial chart that can fill clockwise or counterclockwise. Other shapes of progress tracker are also possible. Every time the patient provides an input in response to an ocular exam, the progress value increases incrementally (by any value between zero and infinity) to represent the patient's progress and allow for the computing device 1140 to conduct an arbitrary number of steps. This progress value is transformed into a fill amount percentage based on an asymptotic formula that approaches, but does not reach, 100%. For example, the formula might be fill amount percentage=100%×(v/(v+1)), where v=progress value/the number of answers required to fill 50% of the progress tracker. Other formulas are also possible. For instance, non-linear and piecewise functions can be used to adjust the speed at which the patient's progress tracker fills.

In some embodiments, the computing device 1140 processes the patient's medical history and biodata in addition to the input from the patient. The medical history and biodata are also compared to the database in order to evaluate the patient for one or more ocular conditions and/or sub-conditions.

In other embodiments, the computing device 1140 implements a machine-learning algorithm to process the input in real-time and decide whether to continue conducting an ocular exam or move onto a subsequent ocular exam.

Optionally, the computing device 1140 implements a machine-learning algorithm to compare the input with one or more databases in real-time. This can include implementing the machine-learning algorithm to evaluate the patient for one or more ocular conditions and/or sub-conditions in order to provide the patient with a diagnosis of one or more ocular conditions and/or sub-conditions.

Optionally, the computing device 1140 recommends a plan for treating the ocular conditions and/or sub-conditions with which the patient is diagnosed. In some embodiments, the computing device 1140 implements a machine-learning algorithm to recommend a tailored treatment plan. In other embodiments, the computing device 1140 analyzes the patient's medical history (including the patient's risk profile and ocular health history) and biodata while developing a treatment plan for the patient.

### Detecting and Treating Eye Misalignment with Dynamic Tasks

Some implementations of the vision evaluation methods described herein comprise detecting and treating various ocular conditions associated with eye misalignment (e.g., strabismus, amblyopia, convergence insufficiency, etc.) by administering visual tasks in a dynamic manner. These detection and treatment methods can be implemented using the vision evaluation system described above with respect to FIGS. 11A-11B, which reduces the invasive nature of these evaluations, as compared to traditional vision evaluation methods. The VR system 1120 is in electronic communication with the computing device 1140. The computing device 1140 is configured to process the data or input collected by the sensors 1126 and use that data or input to control a display on the screens 1124. Controlling the environment displayed on the screens 1124 includes conducting visual tasks in which the patient wearing the VR headset can participate.

### Detecting Eye Misalignment

At least some of the embodiments of the evaluation methods and systems disclosed herein reflect the realization that the traditional method for evaluating a patient's eye coordination necessarily involves human error. In the traditional method, a physician exposes the patient to various visual phenomena and observes the patient for indicators that the patient is perceiving the phenomena. Using the method for evaluating eye coordination described herein, the patient can personally inform the VR system 1120 (or the physician) when he is perceiving the visual phenomena, which minimizes the chance of human error. Because the evaluation is conducted by the VR system 1120, this method is simultaneously less invasive and at least three times more accurate than the traditional method.

An embodiment of the method for detecting eye misalignment described herein begins with the patient wearing the VR headset 1122. In some embodiments, the sensors 1126 and the cameras 1128 on the VR headset 1122 start collecting data about the patient. The data can include biodata (e.g., scarring, skin tone around the patient's eyes, blink rate, pupil diameter, etc.), the patient's previous prescriptions, and the patient's ocular history (e.g., interior segment, potential disease like cataracts, corneal abrasions, scarring, issues with posterior segment, retinopathy, retinal scarring, etc.).

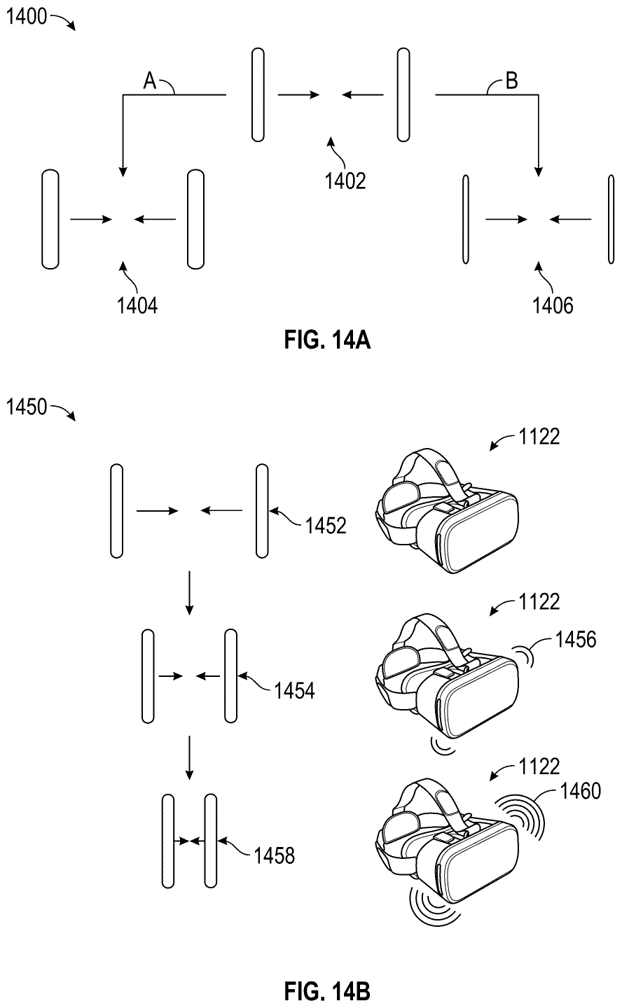

Once the patient is wearing the VR headset 1122, the computing device 1140 causes a first visual task to be conducted on the screens 1124 of the VR headset 1122. The visual task comprises a task that challenges the patient's binocular vision and assess coordination between the patient's eyes by engaging the patient in tasks that require precise eye coordination. The visual task can be a depth perception exercise, an exercise that requires patients to align virtual objects, or an exercise that requires patients to track moving targets in real-time. An example of a visual task is illustrated in FIG. 14A. Visual task 1402 involves two lines moving towards each other and requires the patient to indicate when the two lines converge. In some embodiments, the characteristics of the first visual task are determined by the patient's biodata and medical history. For instance, the patient's biodata and medical history can affect the thickness of the lines in visual task 1402, the speed at which the lines converge, or the brightness at which the visual task 1402 is displayed on the screens 1124. The patient's biodata and medical history can even cause the computing device 1140 to display an entirely different type of visual task.

As the patient participates in the first visual task, the patient provides a first input to the VR headset 1122. For example, the patient can provide voluntary responses in the form of verbal confirmations received by the microphones 1132 of the VR headset 1132 or tactile inputs received by the sensors 1138 on the handheld device 1136. The patient can also provide involuntary responses such as changes in blink rate, heart rate, eye movements, pupil responses, or fixation stability, all of which can be perceived by the sensors 1126 and the cameras 1128 on the VR headset or the sensors 1138 on the handheld device 1136.

The VR headset 1122 collects this first input and shares it with the computing device 1140. The computing device 1140 processes this first input and uses the first input to adapt the first visual task to better suit the patient's needs. This method is illustrated in FIG. 14A, described in greater detail below. Adapting the first visual task entails developing and/or identifying a second visual task with a different difficulty and/or nature than the first visual task. The computing device 1140 then causes the second visual task to be conducted on the screens 1124 of the VR headset 1122, and the VR headset 1122 collects a second input, which will be processed by the computing device 1140.

FIG. 14A illustrates an example of the method of dynamically administering visual tasks to detect eye misalignment, in accordance with some embodiments. In this example of the method 1400, the computing device 1140 causes a visual task 1402 (i.e., the first visual task) to be displayed on the screens 1124 of the VR headset 1122. As described above, the first visual task 1402 involves two lines moving towards each other and requires the patient to indicate when the two lines converge. If the patient fails or struggles to complete the first visual task 1402, then the computing device 1140 follows path A and adapts the first visual task 1402 into a visual task 1404 (i.e., the second visual task), in which the two lines are thicker. Because the patient could not successfully complete the first visual task 1402, the computing device 1140 provides the patient with a difficult second visual task 1404 in order to assess the severity of the patient's eye misalignment.

If the patient successfully completes the first visual task 1402, then the computing device 1140 follows path A and adapts the first visual task 1402 into a visual task 1406 (i.e., the alternative second visual task), in which the two lines are thinner. This adaptation recognizes the patient's ability to successfully complete the first visual task 1402 and provides the patient with a more difficult second visual task 1406 in order to evaluate the patient's eye misalignment more closely and obtain a more nuanced diagnosis. Ordinary vision evaluation methods would simply stop the testing after the first visual task 1402 because the results indicate that eye misalignment does not currently hinder the patient's vision. However, the method described herein continues testing in order to detect smaller symptoms of eye misalignment, which can lead to early diagnoses and preventive treatment.

The computing device 1140 processes the input collected by the VR headset 1122 to evaluate the patient's eye coordination and determine whether the patient has eye misalignment. The first and second input comprises the patient's confirmation that he is perceiving the phenomena displayed (as part of the visual task), failing to perceive the phenomena displayed, or is otherwise participating in the visual task.

The computing device 1140 evaluates the patient's eye coordination by comparing the input to a database of responses from to the visual tasks from individuals with known eye misalignment disorders. In some embodiments, every set of input collected from the patient (e.g., first, second, third, . . . , nth input) is compared to the database. In other embodiments, only the last few sets of input are compared to the database. Optionally, only the last set of input is compared to the database.

In some embodiments, the computing device 1140 implements a multi-layered algorithm to process the input, evaluate the patient's eye coordination, and diagnose the patient with ocular disorders related to eye misalignment. In other embodiments, the multi-layered algorithm processes the input and evaluates the patient's eye coordination in real-time, which facilitates dynamic changes to the characteristics of subsequent visual tasks for a faster and more comprehensive eye misalignment evaluation (as compared to a standard evaluation conducted by a physician).

In some embodiments, the computing device 1140 obtains the input and generates a report of the input. A physician can use the information provided by the report to evaluate the patient. This embodiment of the method of detecting eye misalignment is also faster and more comprehensive than the standard method of evaluating patients for eye misalignment because it utilizes the dynamic adaptation of visual tasks to quickly administer a wide variety of visual tasks.

### Correcting Eye Misalignment

Some embodiments of the eye coordination evaluation methods and systems disclosed herein reflect the realization that dynamically switching between various visual tasks is a more effective way of correcting eye misalignment than traditional therapeutic exercises. The method for correcting eye misalignment begins with the patient wearing the VR headset 1122. In some embodiments, the sensors 1126 and the cameras 1128 on the VR headset 1122 start collecting data about the patient such as biodata (e.g., scarring, skin tone around the patient's eyes, blink rate, pupil diameter, etc.) and the patient's medical history (e.g., previous prescriptions and the patient's ocular history).

Once the patient is wearing the VR headset 1122, the computing device 1140 causes a first visual task to be conducted on the screens 1124 of the VR headset 1122. The visual task comprises a task that challenges the patient's binocular vision and assess coordination between the patient's eyes, similar to the visual tasks utilized in the method for detecting eye misalignment disorders, described above. An example of this visual task is illustrated in FIG. 14A. In some embodiments, the characteristics of the first visual task are determined by the patient's biodata and medical history.

As the patient participates in the first visual task, the patient provides a first input to the VR headset 1122 such as voluntary responses (e.g., verbal confirmations or denials, tactile inputs, etc.). The patient can also provide involuntary responses (e.g., changes in blink rate or heart rate).

The VR headset 1122 collects this first input and shares it with the computing device 1140. The computing device 1140 processes this first input and uses the first input to cause the VR headset 1122 to provide corrective feedback to the patient, determine a difficulty and/or nature of a second visual task to be conducted on the screens 1124, and determine the severity of the patient's eye misalignment and/or the probability of future eye misalignment (which includes worsening or improving eye misalignment).

As described above, the computing device 1140 uses the first input to cause the VR headset 1122 to provide corrective feedback to the patient while the patient completes the visual tasks. The corrective feedback informs the patient when a phenomenon is being displayed as part of the visual task. This helps ensure that the patient is actively participating during the visual task. Additionally, the corrective feedback tells the patient whether he is correctly executing the visual task. Real-time corrective feedback not only keeps the patient engaged with the visual task (which increases the effectiveness of the treatment), but it also guides the patient towards correct responses. This guidance trains the patient to combat eye misalignment and is illustrated in FIG. 14B, described in greater detail below. In some embodiments, the corrective feedback includes visual stimuli on the screens 1124 of the VR headset 1122, haptic stimuli at the vibrating motors 1134 of the VR headset 1122 or the vibrating motors 1139 of the handheld device 1136, and/or auditory stimuli at the speakers 1130 of the VR headset 1122.

FIG. 14B illustrates an example of the method of providing corrective feedback to a patient during visual tasks to correct eye misalignment, in accordance with some embodiments. Throughout the administration of the visual task 1450, the VR headset 1122 and the handheld device 1136 provide corrective feedback to the patient, as instructed by the computing device 1140. For the purposes of simplicity, only the VR headset 1122 is illustrated in FIG. 14B.

At the beginning of the visual task, represented by the first stage 1452, the VR headset 1122 might not provide corrective feedback to the patient. However, as the visual task continues—for example, into the second stage 1454—and the lines move closer together, the VR headset 1122 will provide a first corrective feedback 1456 in the form of visual, haptic, and/or auditory stimuli. As the visual task continues into the third stage 1458 and the lines move even closer, the VR headset 1122 provides a second corrective feedback 1460 to the patient. The second corrective feedback 1460 may have a greater intensity or frequency than the first corrective feedback 1456. In some embodiments, the first corrective feedback 1456 is a beeping noise, and the second corrective feedback 1460 is a louder beeping noise. In other embodiments, the first corrective feedback 1456 is a beeping noise, and the second corrective feedback 1460 is a beeping noise combined with vibrations.

In addition to instructing the VR headset 1122 to provide corrective feedback to the patient, the computing device 1140 determines the difficulty and the nature of the second visual task. Using the patient's responses to the first visual task (i.e., the first input), the computing device 1140 develops the second visual task by adapting the first visual task based on the patient's needs. This can entail adjusting the difficulty of the first visual task to keep the patient engaged as well as identifying a different type of visual task that will target problems that are specific to the patient or home in on a specific portion of the eye.

Finally, the computing device 1140 determines the severity of the patient's eye misalignment and/or the probability of future eye misalignment by comparing the inputs received in response to the visual tasks to a database of responses from to the visual tasks from individuals with known eye misalignment disorders. In some embodiments, every set of input collected from the patient (e.g., first, second, third, . . . , nth input) is compared to the database. In other embodiments, only the last few sets of input are compared to the database. Optionally, only the last set of input is compared to the database.

In some embodiments, the computing device 1140 implements a multi-layered algorithm to process the input and evaluate the patient's eye coordination. In other embodiments, the multi-layered algorithm processes the input and evaluates the patient's eye coordination in real-time (i.e., the multi-layered algorithm processes the input and evaluates eye coordination as the patient provides responses to the visual tasks). This real-time analysis facilitates dynamic changes to the characteristics of the corrective feedback and the subsequent visual tasks (e.g., changes to the nature, frequency, or intensity of the feedback and tasks). These dynamic changes make for a faster and more comprehensive eye misalignment evaluation (as compared to a standard evaluation conducted by a physician).

### Conducting Children's Vision Screening Through Gamified Tests

Some implementations of the vision evaluation methods dynamically conduct visual tests and examinations in the form of interactive games to diagnose vision problems for patients with short attention spans and patients who have trouble complying with vision exams. For example, this method can be used for evaluating children for various pediatric vision problems (e.g., amblyopia, strabismus, refractive errors, etc.) by administering visual tests designed to assess visual acuity, depth perception, color vision, eye coordination, and other aspects of visual health. These methods can be implemented using a vision evaluation system like the one described with respect to FIGS. 11A-11B.

The VR system 1120 is in electronic communication with the computing device 1140. The computing device 1140 is configured to process the data or input collected by the sensors 1126 and use that data or input to control an environment displayed on the screens 1124. Controlling the environment displayed on the screens 1124 includes administering gamified visual tests and dynamically changing the gamified visual tests being administered based on the patient's responses to the gamified visual tests. The computing device 1140 continues to change the gamified visual test in order to keep the patient engaged with the evaluation until the patient has been evaluated for all relevant ocular conditions.

In some embodiments, the computing device 1140 includes a user interface 1142. At this user interface 1142, the patient, the patient's physician, or the patient's parent can access the results of the evaluation. Additionally, the user interface 1142 can receive inputs from the patient, the patient's physician, or the patient's parent. These inputs can change the direction or difficulty of the evaluation or provide the computing device 1140 with additional information such as medical history or behavioral observations.

At least some of the embodiments disclosed herein reflect the realization that physicians have difficulty accurately diagnosing patients who have short attention spans and/or have trouble complying with vision exams (e.g., children). By formatting the visual tests as games, tailoring the visual test to the child's age and developmental stage, and dynamically adjusting the difficulty of the visual test based on the child's performance, the method disclosed herein keeps the child engaged throughout the evaluation process. This results in diagnoses that are at least two times more accurate than diagnoses given by a standard practitioner with five years of experience. Moreover, this method conducts a comprehensive evaluation at least two times faster than a standard clinician with five years of experience administering the visual tests required to conduct a similar evaluation. This is accomplished by obviating the need for the child to reach the end of any given visual test by collecting the child's responses to the visual test in real-time, analyzing those responses, and utilizing those response to transition into a subsequent visual test. The difficulty and nature of the subsequent visual test is based on the child's responses to the initial visual test. Not only does this method enable the child to participate in a multitude of visual tests in a shortened period of time (which makes the best use of a child's short attention span and reduces the cost of associated medical bills by reducing the number of appointments required for a complete evaluation), but this method also leads to a narrower and more nuanced diagnosis because it facilitates a more comprehensive evaluation.

The process starts with the child donning the VR headset 1122, and the sensors 1126 on the VR headset 1122 start collecting data about the child. The data can include biodata such as heart rate, blink rate, pupil diameter, etc., as well as the child's medical history. The computing device 1140 processes this data in order to determine the type and difficulty of the gamified visual tests administered on the screens 1124 of the VR headset 1122. For instance, the computing device 1140 processes this data to evaluate the degree to which the child is engaging with the gamified visual test as well as the child's stress levels during the gamified visual test. The child's engagement and stress levels are evaluated throughout the administration of the various gamified visual tests to suggest an accuracy of the visual health evaluation by indicating whether the child's responses are driven by factors affecting the child's visual health or by external factors (e.g., being distracted, disengaged, or stressed).

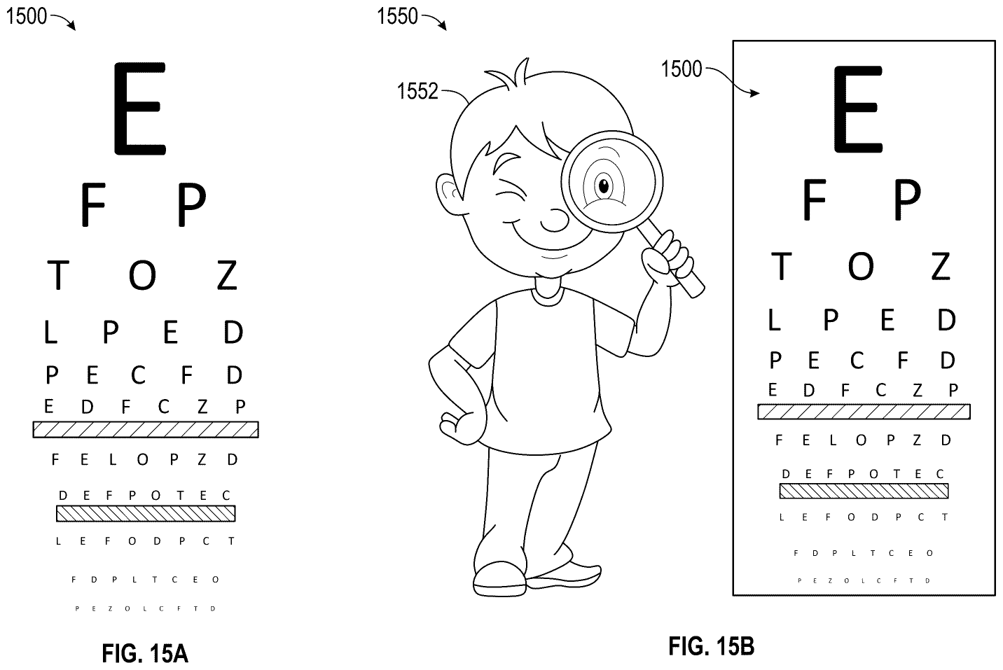

Examples of the types and difficulties of visual tests that can be administered during this method and the logic tree that determines the order in which the visual tests are displayed have been described above with respect to FIGS. 13A-13C. An example of the visual test being gamified for children and displayed in an interactive, immersive environment is illustrated in FIGS. 15A-15B.

FIGS. 15A-15B illustrate a traditional method of administering a visual test and a gamified method of administering the visual test for children, in accordance with some embodiments. FIG. 15A shows a traditional visual test 1500 that can be difficult for a child to engage with. FIG. 15B shows a gamified visual test 1550, which has the traditional visual test 1500 displayed in an immersive cartoon environment with cartoon character 1552 talking the child through the visual test to help keep the child engaged. This gamified environment also helps keep the child relaxed by avoiding the clinical, serious nature of traditional visual tests.

The child participates in the first gamified visual test and provides a response in the form of a first input. The first input comprises both voluntary and involuntary responses from the child. These voluntary and involuntary responses can be audio, visual, and tactile inputs from the patient (e.g., verbal confirmations, blink rates, and button pushing) received at the sensors 1126 and 1138, the microphones 1132, or the cameras 1128.

After the first input is collected at the VR headset 1122, the computing device 1140 processes the first input. This processing can include evaluating the degree to which the child is engaged with the first visual test as well as the child's stress levels while completing the first visual test. Moreover, processing the first input can also include comparing the first input with a database that includes input from a group of individuals with known pediatric vision problems. In some embodiments, an artificial intelligence processes the first input to evaluate engagement and stress or to compare the first input to the database. Based on the child's level of engagement, the child's level of stress, and how the child's responses compare to responses in the database, the computing device 1140 determines the characteristics (i.e., nature, intensity, difficulty, duration, etc.) of the second gamified visual test administered on the screens 1124 of the VR headset 1122. Optionally, an artificial intelligence determines the characteristics of the second gamified visual test. The artificial intelligence can process the first input to tailor the characteristics of the second gamified visual tests to the child's immediate needs (e.g., increasing the difficulty of the test for greater engagement or changing the type of test to test for different ocular conditions).

The child next participates in the second gamified visual test and provides a response in the form of a second input. As described above with respect to the first input, the sensors 1126 and 1138, the microphones 1132, the cameras 1128, or the vibrating motors 1134 and 1139 collect the second input, so that the computing device 1140 can process the second input. Depending on the characteristics of the second input as compared to the input in the database, the computing device 1140 can continue administering the second gamified visual test or administer a third gamified visual test with a different type and/or difficulty. This dynamic, iterative process of administering gamified visual tests continues to provide the child with increasingly tailored gamified tests until the computing device 1140 has collected enough information to complete a comprehensive ocular evaluation.

In some embodiments, the computing device 1140 processes the data and the input in real-time during administration of the gamified visual tests. Continuously processing the data facilitates dynamic changes to the characteristics of subsequent gamified visual tests for a faster and more comprehensive evaluation (as compared to a standard evaluation conducted by a physician). Optionally, the computing device 1140 implements an artificial intelligence program to process the data and the input in real-time.

The computing device 1140 uses the data and the input (which can include every set of input, the last few sets of input, or the last set of input) to evaluate the child for ocular conditions. In some embodiments, the computing device 1140 compares the data and the input with a database to evaluate the child for one or more pediatric vision problems. The database comprises data and input from a control group of children (i.e., children with known visual health statuses). Optionally, artificial intelligence is used to compare the data and the input with the database.

In some embodiments, the computing device 1140 produces a report detailing the gamified visual tests administered as well as the data and input received during the evaluation. The report may comprise a summary of the child's visual health, recommendations for further ocular testing, and recommendations for corrective measures or treatment. In other embodiments, the computing device 1140 continuously outputs the report as the child completes the gamified visual tests. The report is accessible via the user interface 1142.

Optionally, a third party (e.g., a physician or the child's parent) can provide inputs at the user interface 1142 to change the difficulty of the gamified visual tests or redirect the nature of the gamified visual tests. For example, if a parent notices that the child struggles to perceive different colors, the parent can provide this information at the user interface 1142 to redirect the gamified visual tests to test the child for color blindness.

### Detecting and Treating Visual Processing Disorders With Multisensory Integration

Some implementations of the vision evaluation methods disclosed herein comprise utilizing multisensory integration to provide dynamic feedback to a patient while the patient undergoes evaluations and treatment for visual processing disorders (e.g., dyslexia, visual-motor integration issues, etc.). These methods can be implemented using a vision evaluation system like the one described above with respect to FIGS. 11A-11B, which makes these methods far less invasive and less time consuming than traditional vision evaluation methods.

The VR system 1120 is in electronic communication with the computing device 1140. The computing device 1140 is configured to process the data or input collected by the sensors 1126 and use that data or input to control an environment displayed on the screens 1124. Controlling the environment displayed on the screens 1124 includes conducting a visual task through the screens 1124 of the VR headset 1122 and reconducting the visual task with feedback based on discrepancies in the patient's visual perception, discrepancies in the patient's responses to the visual tasks, and the severity of the patient's visual processing disorder.

This method uses the VR system 1120 to monitor the patient's eye movements, reaction times, and coordination across multiple sensory modalities, which provides comprehensive data that can be used to diagnose visual processing disorders. This method of holistically evaluating the patient for visual processing disorders provides significantly more data than the traditional method of diagnosing visual processing disorders without requiring on the large, cumbersome machinery conventionally associated with holistic evaluations. As a result, this method leads to narrower, more nuanced diagnoses while relying on a simple, compact VR system 1120 to collect holistic data. Moreover, real-time adaptation of the stimuli in the environment and/or the characteristics of the visual task also facilitates a more comprehensive evaluation than that conducted by a standard physician in the traditional manner because the patient can undergo a wider range of visual tasks in a shorter period of time.

### Detecting Visual Processing Disorders

When the patient puts on the VR headset 1122, the VR headset 1122 starts collecting data about and input from the patient and continues to collect data and input throughout the evaluation method. The data can include the patient's physiological responses (e.g., pupil dilation, blink rates, heart rate, skin conductance, etc.) throughout the duration of the evaluation. Both the data and the input can include the patient's multisensory integration patterns throughout the duration of the evaluation. The computing device 1140 processes the data and input and uses it to develop an environment. Then, the computing device 1140 causes the environment to be displayed on the screens 1124 of the VR headset 1122. The environment can also include auditory stimuli that can be projected from the speakers 1130 of the VR headset 1122 and tactile stimuli that can be provided at the vibrating motors 1134 of the VR headset 1122 or the handheld device 1136. The computing device 1140 causes a visual task to be conducted on the VR headset 1122 within this environment.

The patient responds to the visual task by providing a first input to the VR headset 1122 and/or the handheld device 1136. The computing device 1140 processes the first input to search for discrepancies between the patient's visual perception and the environment displayed on the screens 1124. In some embodiments, an adaptation algorithm processes the first input to search for discrepancies. Optionally, the first input is processed in real-time by the computing device 1140 or the adaptation algorithm.

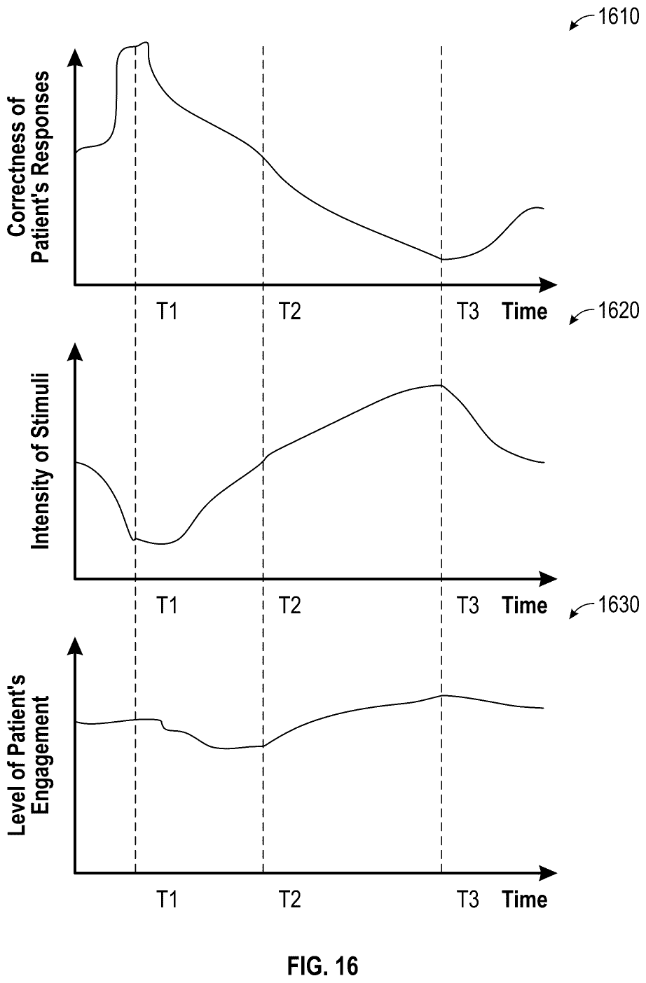

Based on the discrepancies identified, the computing device 1140 causes the visual task to be reconducted on the VR headset 1122. The visual task can be reconducted while the computing device provides feedback to the patient. This feedback can include adjustments to the intensity and/or nature of the visual stimuli (e.g., increased brightness or saturation), auditory stimuli (e.g., beeping noises or increased volume), and/or tactile stimuli (e.g., vibrations or buzzing). In some embodiments, the intensity and/or nature of the visual, auditory, and/or tactile stimuli is determined by an adaptation algorithm processing the patient's input. In other embodiments, the patient's input is processed in real-time, and the feedback is adapted in real-time in order to help maintain the patient's engagement with the visual task. The relationship between the patient's input, the intensity of the stimuli, and the patient's level of engagement is discussed in greater detail with respect to FIG. 16.

FIG. 16 illustrates the relationship between the correctness of the patient's responses to a visual task, the intensity of the stimuli provided in the immersive VR environment during the visual task, and the patient's level of engagement during the visual task, in accordance with some embodiments. As the patient undergoes the evaluation for visual processing disorders and provides responses, the computing device 1140 provides feedback to the patient through the VR system 1120. The feedback is synchronized with the visual task and informs the patient when the visual task requires input. This ensures that the patient remains engaged with the visual task, which increases the accuracy of the evaluation by confirming that the patient's incorrect answers or unresponsiveness are due to the patient's inability to perceive phenomenon on the screen 1124 rather than the patient's lack of engagement.

In the example shown in FIG. 16, the patient provides correct response at time T1 as shown by the peak in the graph 1610. Because the patient is answering correctly, the intensity of the stimuli (i.e., the feedback) in the environment decreases, as shown by the dip at time T1 in the graph 1620. At time T2, the graph 1610 indicates that the patient is providing incorrect responses to the visual task, which correlate to a dip in the patient's level of engagement, as shown in the graph 1630. In response to the incorrect responses and the decreases engagement, the intensity of the stimuli increases, as shown by the upward slope in the graph 1620 at T2. The intensity of the stimuli increases until the patient starts providing correct responses and the patient's engagement level increases at T3.

While experiencing the adjustments in stimuli, the patient responds to the visual task and provides a second input to the VR headset 1122 and/or the handheld device 1136. The computing device 1140 processes the second input to search for discrepancies and causes the visual task to be reconducted with adjusted feedback based on the discrepancies identified. This process continues until the computing device gathers enough data and input to evaluate the patient comprehensively for visual processing disorders.

In some embodiments, the adaptation algorithm processes the second input to search for discrepancies. Optionally, the second input is processed in real-time by the computing device 1140 or the adaptation algorithm.

As the computing device 1140 gathers data and input, the computing device 1140 compares the data and input with a database of data and input from individuals with known visual processing disorders. This comparison guides the computing device 1140 to diagnose the patient with one or more visual processing disorders. Optionally, the adaptation algorithm compares the data and the input with the database. In other embodiments, the data and the input are processed in real-time by the computing device 1140 or the adaptation algorithm.

In some embodiments, every set of input collected from the patient (e.g., first, second, third, . . . , nth input) is compared to the database. In other embodiments, only the last few sets of input are compared to the database. Optionally, only the last set of input is compared to the database.

### Treating Visual Processing Disorders

When the patient puts on the VR headset 1122, the VR headset 1122 starts collecting data about and input from the patient and continues to collect data and input throughout the evaluation method. The data can include the patient's physiological responses (e.g., pupil dilation, blink rates, heart rate, skin conductance, etc.) throughout the duration of the evaluation. Both the data and the input can include the patient's multisensory integration patterns throughout the duration of the evaluation. The computing device 1140 processes the data and input and uses it to develop an environment. Then, the computing device 1140 causes the environment to be displayed on the screens 1124 of the VR headset 1122. The environment can also include auditory stimuli that can be projected from the speakers 1130 of the VR headset 1122 and tactile stimuli that can be provided at the vibrating motors 1134 of the VR headset 1122 or the handheld device 1136. The computing device 1140 causes a first visual task to be conducted on the VR headset 1122 within this environment.

The patient responds to the first visual task by providing a first input to the VR headset 1122 and/or the handheld device 1136. The computing device 1140 processes the first input to determine the severity of the patient's visual processing disorder. The severity is dynamic and can decrease as the patient continues to use the VR system for therapy or increase as the patient takes breaks from treatment. Optionally, an adaptation algorithm processes the first input to determine the severity of the patient's visual processing disorder. In some embodiments, the first input is processed in real-time by the computing device 1140 or the adaptation algorithm.

Based on the severity identified, the computing device 1140 causes a second visual task to be conducted on the VR headset 1122. The second visual task is tailored to treat the patient's visual processing disorder and comprises a different difficulty and/or nature than the first visual task. For example, a more severe visual processing disorder might correlate with a more intense visual task. In some embodiments, an adaptation algorithm determines the difficulty and/or nature of the second visual task. In other embodiments, the difficulty and/or nature of the second visual task is determined in real-time by the computing device 1140 or the adaptation algorithm.

The second visual task can also be conducted with an adjusted environment (i.e., adjusted visual, auditory, and/or tactile stimuli). The adjustments to the visual, auditory, and/or tactile stimuli can be based on the accuracy of the patient's input and/or the patient's level of engagement as described above with respect to FIG. 16.

The patient responds to the second visual task and provides a second input to the VR headset 1122 and/or the handheld device 1136. The computing device 1140 processes the second input to determine the severity of the patient's visual processing disorder at this stage and cause a third visual task to be conducted. This process can continue for a duration of time set (e.g., by the patient or his physician) at the user interface 1142 of the computing device 1140. In some embodiments, this process continues over multiple sessions, and each session is optimized by the computing device to treat the patient's visual processing disorder based on the nature and severity of the visual processing disorder.

### Illustration of Subject Technology As Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method of displaying visual anomalies for diagnosing an ocular condition, the method comprising: collecting data about or input from a patient wearing a virtual reality (VR) headset; displaying a first visual anomaly on a screen of the VR headset; collecting a first input regarding a response from the patient to the first visual anomaly; displaying a second visual anomaly on the screen; collecting a second input regarding a response from the patient to the second visual anomaly; and based on at least one of the first or second input, evaluating the patient for an ocular condition.

Clause 2. The method of Clause 1, wherein the data comprises a presence of scarring.

Clause 3. The method of any of the preceding Clauses, wherein the data comprises a tone of skin surrounding an eye.

Clause 4. The method of any of the preceding Clauses, wherein the data comprises a blink rate.

Clause 5. The method of any of the preceding Clauses, wherein the data comprises a pupil diameter.

Clause 6. The method of any of the preceding Clauses, wherein the input comprises a patient's reaction time.

Clause 7. The method of any of the preceding Clauses, wherein the input comprises a patient's eye movements.

Clause 8. The method of any of the preceding Clauses, wherein the collecting data comprises using at least one sensor of the VR headset to collect the data about or input from the patient.

Clause 9. The method of any of the preceding Clauses, wherein the displaying the first visual anomaly comprises displaying the first visual anomaly based on the data about or input from the patient.

Clause 10. The method of any of the preceding Clauses, wherein the evaluating comprises comparing at least one of the first or second input with a database to evaluate the patient for an ocular condition.

Clause 11. The method of any of the preceding Clauses, wherein the evaluating comprises comparing the first input with a database to evaluate the patient for an ocular condition.

Clause 12. The method of any of the preceding Clauses, wherein the evaluating comprises comparing the second input with a database to evaluate the patient for an ocular condition.

Clause 13. The method of any of the preceding Clauses, wherein the first and second visual anomalies comprise blurred vision.

Clause 14. The method of any of the preceding Clauses, wherein the first and second visual anomalies comprise double vision.

Clause 15. The system of any of the preceding Clauses, wherein the first and second visual anomalies comprise floaters.

Clause 16. The method of any of the preceding Clauses, wherein the first and second visual anomalies comprise field loss.

Clause 17. The method of any of the preceding Clauses, wherein the second visual anomaly has a different intensity than the first visual anomaly.

Clause 18. The method of any of the preceding Clauses, wherein the second visual anomaly has a different frequency than the first visual anomaly.

Clause 19. The method of any of the preceding Clauses, wherein the second visual anomaly has a different nature than the first visual anomaly.

Clause 20. A system for simulating visual anomalies to diagnose ocular conditions, the system comprising: a virtual reality (VR) headset having a screen and at least one sensor configured to collect data about or input from a patient wearing the VR headset; and a computing device in electronic communication with the VR headset and being configured to process the data or input from the patient and control an environment displayed on the screen based on the data or input, wherein the computing device is configured (i) to cause a first visual anomaly to be displayed on the screen and to receive a first input from the VR headset in response to the first visual anomaly, (ii) to cause a second visual anomaly to be displayed on the screen and to receive a second input from the VR headset in response to the second visual anomaly, and (iii) to compare at least one of the first or second input with a database for diagnosing the patient for an ocular condition.

Clause 21. The method of Clause 20, wherein the data comprises a presence of scarring.

Clause 22. The method of any of Clauses 20 to 21, wherein the data comprises a tone of skin surrounding an eye.

Clause 23. The method of any of Clauses 20 to 22, wherein the data comprises a blink rate.

Clause 24. The method of any of Clauses 20 to 23, wherein the data comprises a pupil diameter.

Clause 25. The system of any of Clauses 20 to 24, wherein the input, the first input, and the second input comprise a patient's reaction time.

Clause 26. The system of any of Clauses 20 to 25, wherein the input, the first input, and the second input comprise a patient's eye movements.

Clause 27. The system of any of Clauses 20 to 26, wherein the input, the first input, and the second input comprise a patient's blink rate.

Clause 28. The system of any of Clauses 20 to 27, wherein the input, the first input, and the second input comprise a patient's pupil dilation.

Clause 29. The system of any of Clauses 20 to 28, wherein the environment comprises a visual display.

Clause 30. The system of any of Clauses 20 to 29, wherein the computing device causes the first visual anomaly to be displayed in a first environment.

Clause 31. The system of any of Clauses 20 to 30, wherein the computing device causes the second visual anomaly to be displayed in a second environment, different from the first environment.

Clause 32. The system of any of Clauses 20 to 31, wherein the computing device causes the second visual anomaly to be displayed based on the first input.

Clause 33. The system of any of Clauses 20 to 32, wherein the computing device is configured to compare the first and second input to the database for diagnosing an ocular condition.

Clause 34. The system of any of Clauses 20 to 33, wherein the computing device is configured to compare the first input to the database for diagnosing an ocular condition.

Clause 35. The system of any of Clauses 20 to 34, wherein the computing device is configured to compare the second input to the database for diagnosing an ocular condition.

Clause 36. The system of any of Clauses 20 to 35, wherein the at least one sensor is configured to collect the first input in response to the first visual anomaly.

Clause 37. The system of any of Clauses 20 to 36, wherein the at least one sensor is configured to collect the second input in response to the second visual anomaly.

Clause 38. The system of any of Clauses 20 to 37, wherein the first and second visual anomalies comprise blurred vision.

Clause 39. The system of any of Clauses 20 to 38, wherein the first and second visual anomalies comprise double vision.

Clause 40. The system of any of Clauses 20 to 39, wherein the first and second visual anomalies comprise floaters.

Clause 41. The system of any of Clauses 20 to 40, wherein the first and second visual anomalies comprise field loss.

Clause 42. The system of any of Clauses 20 to 41, wherein the second visual anomaly has a different intensity than the first visual anomaly.

Clause 43. The system of any of Clauses 20 to 42, wherein the second visual anomaly has a different frequency than the first visual anomaly.

Clause 44. The system of any of Clauses 20 to 43, wherein the second visual anomaly has a different nature than the first visual anomaly.

Clause 45. The system of any of Clauses 20 to 44, wherein the second visual anomaly is a more accurate simulation of what the patient perceives than the first visual anomaly.

Clause 46. The system of any of Clauses 20 to 45, wherein a machine learning algorithm processes the first input at the computing device to determine an extent to which an intensity or a frequency of the second visual anomaly must differ from the first visual anomaly or to determine whether the second visual anomaly must be of a different nature than the first visual anomaly.

Clause 47. The system of any of Clauses 20 to 46, wherein a machine learning algorithm processes the second visual anomaly at the computing device to evaluate the patient for an ocular condition.

Clause 48. The system of any of Clauses 20 to 47, wherein a physician processes the first input at the computing device to determine an extent to which an intensity or a frequency of the second visual anomaly must differ from the first visual anomaly or to determine whether the second visual anomaly must be of a different nature than the first visual anomaly.

Clause 49. The system of any of Clauses 20 to 48, wherein a physician processes the second visual anomaly at the computing device to evaluate the patient for an ocular condition.

Clause 50. A method of dynamically administering ocular exams, the method comprising: conducting a first ocular exam on a screen of a virtual reality (VR) headset to evaluate a patient wearing the VR headset for a first ocular condition; receiving a first input from the patient; comparing the first input with a database to determine whether to diagnose the patient with the first ocular condition; after comparing the first input with the database, continuing to conduct the first ocular exam and receive the first input; conducting a first ocular sub-exam on the screen to evaluate the patient for a first ocular sub-condition and receiving a first sub-input from the patient; or conducting a second ocular exam on the screen to evaluate the patient for a second ocular condition and receiving a second input from the patient.

Clause 51. The method of Clause 50, wherein the method further comprises, before conducting the first ocular sub-exam or conducting the second ocular exam, ending the first ocular exam before the patient completes the first ocular exam.

Clause 52. The method of any of Clauses 50 to 51, wherein the method further comprises, before conducting the first ocular exam, receiving a medical history and biodata of the patient.

Clause 53. The method of any of Clauses 50 to 52, further comprising processing the medical history and biodata to determine a nature of the first ocular exam.

Clause 54. The method of any of Clauses 50 to 53, wherein comparing the first input comprises implementing a machine-learning algorithm to compare the first input with the database.

Clause 55. The method of any of Clauses 50 to 54, further comprising implementing a machine-learning algorithm to process the first input, the first sub-input, and the second input to determine the nature of a next ocular exam or sub-exam.

Clause 56. The method of any of Clauses 50 to 55, further comprising implementing a machine-learning algorithm to process the first input, the first sub-input, and the second input to diagnose the patient with one or more of a first ocular condition, a first ocular sub-condition, or a second ocular condition.

Clause 57. The method of any of Clauses 50 to 56, further comprising diagnosing the patient with one or more of the first ocular condition, the first ocular sub-condition, or the second ocular condition.

Clause 58. The method of any of Clauses 50 to 57, further comprising recommending a plan to treat the first ocular condition, the first ocular sub-condition, or the second ocular condition with which the patient is diagnosed.

Clause 59. A system for dynamically administering ocular exams, the system comprising: a virtual reality (VR) headset having a screen and at least one sensor configured to collect input from a patient wearing the VR headset; and a computing device in electronic communication with the VR headset and being configured to process the input from the patient and control an environment displayed on the screen based on the input, wherein the computing device is configured to (a) conduct a first ocular exam on the screen to evaluate the patient for a first ocular condition and receive a first input from the VR headset; (b) compare the first input with a database for diagnosing the patient with the first ocular condition; and (c)(i) continue to conduct the first ocular exam and receive the first input, (ii) conduct a first ocular sub-exam on the screen to evaluate the patient for a first ocular sub-condition and receive a first sub-input from the VR headset, or (iii) conduct a second ocular exam on the screen to evaluate the patient for a second ocular condition and receive a second input from the VR headset.

Clause 60. The system of Clause 59, wherein the VR headset further comprises cameras, speakers, and microphones.

Clause 61. The system of any of Clauses 59 to 60, wherein the first input comprises an audio input received at the microphones.

Clause 62. The system of any of Clauses 59 to 61, wherein the first input comprises a visual input received at the sensors or the cameras.

Clause 63. The system of any of Clauses 59 to 62, wherein the visual input comprises a blink rate.

Clause 64. The system of any of Clauses 59 to 63, wherein the visual input comprises a pupil dilation.

Clause 65. The system of any of Clauses 59 to 64, wherein the visual input comprises saccades.

Clause 66. The system of any of Clauses 59 to 65, wherein the visual input comprises smooth pursuit movements.

Clause 67. The system of any of Clauses 59 to 66, wherein the visual input comprises vergence movements.

Clause 68. The system of any of Clauses 59 to 67, wherein the visual input comprises vestibulo-ocular movements.

Clause 69. The system of any of Clauses 59 to 68, further comprising a handheld device in electronic communication with the VR headset and computing device, and wherein the first input comprises a tactile input received at the handheld device.

Clause 70. The system of any of Clauses 59 to 69, wherein the first input comprises voluntary and involuntary responses by the patient to the first ocular exam.

Clause 71. The system of any of Clauses 59 to 70, wherein the first ocular sub-condition comprises a symptom of the first ocular condition.

Clause 72. The system of any of Clauses 59 to 71, wherein the computing device is further configured to implement a machine-learning algorithm that processes the input in real-time.

Clause 73. The system of any of Clauses 59 to 72, wherein the computing device is further configured to compare all of the input received with one or more databases to evaluate the patient for one or more ocular conditions.

Clause 74. The system of any of Clauses 59 to 73, wherein the computing device is further configured to compare the first input and the first sub-input with one or more databases to evaluate the patient for the first ocular condition and the first ocular sub-condition.

Clause 75. The system of any of Clauses 59 to 74, wherein the computing device ends the first ocular exam and conducts the first ocular sub-exam or the second ocular exam before the patient completes the first ocular exam.

Clause 76. The system of any of Clauses 59 to 75, wherein the computing device is further configured to receive a medical history and biodata of the patient and implement a machine-learning algorithm to process the medical history and biodata and determine whether to diagnose the patient with an ocular condition.

Clause 77. The system of any of Clauses 59 to 76, wherein the computing device is further configured diagnose the patient with one or more ocular conditions or ocular sub-conditions.

Clause 78. The system of any of Clauses 59 to 77, wherein the computing device is further configured to implement a machine-learning algorithm to process a diagnosis, a medical history, and biodata in order to recommend a treatment of the ocular condition.

Clause 79. The system of any of Clauses 59 to 78, wherein the computing device is further configured to (a) receive a second input from the VR headset; (b) compare the second input with a database for diagnosing the patient with the second ocular condition; and (c) (i) continue to conduct the second ocular exam and receive the second input, (ii) conduct a second ocular sub-exam on the screen to evaluate a second ocular sub-condition and receive a second sub-input from the VR headset, or (iii) conduct a third ocular exam on the screen to evaluate a third ocular condition and receive a third input from the VR headset.

Clause 80. A method for detecting eye misalignment, the method comprising: conducting a first visual task on a screen of a virtual reality (VR) headset worn by a patient; collecting a first input regarding a response from the patient to the visual task; conducting a second visual task on the screen; collecting a second input regarding the response from the patient to the second visual task, wherein the second visual task comprises a different difficulty or nature than the first visual task; based on at least one of the first or second input, evaluating the patient's eye coordination to determine whether the patient has eye misalignment.

Clause 81. The method of Clause 80, wherein the method further comprises, before conducting the first visual task, receiving a medical history and biodata of the patient.

Clause 82. The method of any of Clauses 80 to 81, further comprising processing the medical history and biodata to determine a difficulty of the first visual task.

Clause 83. The method of any of Clauses 80 to 82, further comprising processing the medical history and biodata to determine a nature of the first visual task.

Clause 84. The method of any of Clauses 80 to 83, wherein conducting the first and second visual tasks comprises challenging a patient's binocular vision and assessing coordination between a patient's eyes.

Clause 85. The method of any of Clauses 80 to 84, wherein collecting the first and second input comprises evaluating an eye coordination of the patient.

Clause 86. The method of any of Clauses 80 to 85, further comprising implementing a multi-layered algorithm to process the first and second input and evaluate an eye coordination of the patient.

Clause 87. The method of any of Clauses 80 to 86, wherein the multi-layered algorithm processes the first and second input and evaluates the eye coordination in real-time.

Clause 88. The method of any of Clauses 80 to 87, further comprising implementing a multi-layered algorithm to determine the different difficulty of the second visual task.

Clause 89. The method of any of Clauses 80 to 88, further comprising implementing a multi-layered algorithm to determine the different nature of the second visual task.

Clause 90. The method of any of Clauses 80 to 89, wherein evaluating the patient's eye coordination comprises determining a degree of eye misalignment.

Clause 91. The method of any of Clauses 80 to 90, further comprising diagnosing the patient with eye misalignment.

Clause 92. The method of any of Clauses 80 to 91, wherein diagnosing the patient with eye misalignment comprises implementing a multi-layered algorithm to compare the first or second input to a database to evaluate the patient for eye misalignment.

Clause 93. The method of any of Clauses 80 to 92, wherein diagnosing the patient with eye misalignment comprises producing a report of the first or second input for analysis by a physician.

Clause 94. A system for detecting eye misalignment, the system comprising: a virtual reality (VR) headset having a screen and at least one sensor configured to collect input from a patient wearing the VR headset; and a computing device in electronic communication with the VR headset and being configured to process the input from the patient and control a display on the screen, wherein the computing device is configured to (a) conduct a first visual task on the screen and receive a first input from the VR headset in response to the first visual task, (b) conduct a second visual task on the screen and receive a second input from the VR headset in response to the second visual task, and (c) determine whether the patient has eye misalignment.

Clause 95. The system of Clause 94, wherein the first input and the second input comprise voluntary and involuntary responses from the patient.

Clause 96. The system of any of Clauses 94 to 95, wherein the first input and the second input comprise one or more of eye movements, pupil responses, and fixation stability.

Clause 97. The system of any of Clauses 94 to 96, wherein the VR headset further comprises one or more of vibrating motors, sensors, cameras, speakers, and microphones.

Clause 98. The system of any of Clauses 94 to 97, further comprising a handheld device in electronic communication with the VR headset and the computing device, the handheld device having vibrating motors or sensors.

Clause 99. The system of any of Clauses 94 to 98, wherein the patient's responses comprise one or more of visual input received at the sensors or cameras, audio input received at the microphones, and tactile input received at the handheld device.

Clause 100. A method for correcting eye misalignment, the method comprising: conducting a first visual task on a screen of a virtual reality (VR) headset worn by a patient; collecting a first input regarding a response from the patient to the visual task; providing corrective feedback to the patient; determining a severity and probability of future eye misalignment for the patient; and conducting a second visual task on the screen, wherein the second visual task comprises a different difficulty or nature than the first visual task.

Clause 101. The method of Clause 100, wherein the method further comprises, before conducting the first visual task, receiving a medical history and biodata of the patient.

Clause 102. The method of any of Clauses 100 to 101, further comprising processing the medical history and biodata to determine a difficulty of the first visual task.

Clause 103. The method of any of Clauses 100 to 102, further comprising processing the medical history and biodata to determine a nature of the first visual task.

Clause 104. The method of any of Clauses 100 to 103, wherein conducting the first and second visual tasks comprises strengthening binocular vision and preventing progression of misalignment.

Clause 105. The method of any of Clauses 100 to 104, wherein providing corrective feedback comprises providing visual stimuli on the screen.

Clause 106. The method of any of Clauses 100 to 105, wherein providing corrective feedback comprises providing haptic stimuli at vibrating motors positioned on the VR headset.

Clause 107. The method of any of Clauses 100 to 106, wherein providing corrective feedback comprises providing auditory stimuli on the speakers positioned on the VR headset.

Clause 108. The method of any of Clauses 100 to 107, further comprising evaluating a patient's eye coordination by identifying discrepancies in a patient's eye movements.

Clause 109. The method of any of Clauses 100 to 108, further comprising implementing a multi-layered algorithm to process the first and second input and evaluate an eye coordination of the patient.

Clause 110. The method of any of Clauses 100 to 109, wherein the multi-layered algorithm processes the first and second input and evaluates the eye coordination in real-time.

Clause 111. The method of any of Clauses 100 to 110, further comprising implementing a multi-layered algorithm to determine the different difficulty of the second visual task.

Clause 112. The method of any of Clauses 100 to 111, wherein the multi-layered algorithm determines the different difficulty of the second visual task in real-time.

Clause 113. The method of any of Clauses 100 to 112, wherein determining the different difficulty of the second visual task comprises tailoring the different difficulty of the second visual task to the patient based on the severity and probability of future eye misalignment.

Clause 114. The method of any of Clauses 100 to 113, further comprising implementing a multi-layered algorithm to determine the different nature of the second visual task.

Clause 115. The method of any of Clauses 100 to 114, wherein the multi-layered algorithm determines the different nature of the second visual task in real-time.

Clause 116. The method of any of Clauses 100 to 115, wherein determining the different nature of the second visual task comprises tailoring the different nature of the second visual task to the patient based on the severity and probability of future eye misalignment.

Clause 117. A system for correcting eye misalignment, the system comprising: a virtual reality (VR) headset having a screen and at least one sensor configured to collect input from a patient wearing the VR headset; and a computing device in electronic communication with the VR headset and being configured to process the input from the patient and control a display on the screen, wherein the computing device is configured to (a) conduct a visual task on the screen and receive a first input from the VR headset in response to the visual task, (b) cause the VR headset to provide corrective feedback to the patient, (c) determine a severity and probability of future eye misalignment for the patient, and (d) conduct a different visual task on the screen.

Clause 118. The system of Clause 117, wherein the first and second input comprise voluntary and involuntary responses from the patient.

Clause 119. The system of any of Clauses 117 to 118, wherein the first and second input comprise one or more of eye movements, pupil responses, and fixation stability.

Clause 120. The system of any of Clauses 117 to 119, wherein the VR headset further comprises one or more of vibrating motors, sensors, cameras, speakers, and microphones.

Clause 121. The system of any of Clauses 117 to 120, further comprising a handheld device in electronic communication with the VR headset and the computing device, the handheld device having vibrating motors or sensors.

Clause 122. The system of any of Clauses 117 to 121, wherein the patient's responses comprise one or more of visual input received at the sensors or cameras, audio input received at the microphones, and tactile input received at the handheld device.

Clause 123. A method for conducting children's vision screening, the method comprising: collecting data about or input from a child wearing a virtual reality (VR) headset; administering a first gamified visual test on a screen of the VR headset; collecting a first input regarding a response from the child to the first gamified visual test; administering a second gamified visual test on the screen; collecting a second input regarding a response from the child to the second gamified visual test; and based on at least one of the first or second input, evaluating a child's visual health.

Clause 124. The method of Clause 123, wherein collecting the data about the child comprises using sensors on the VR headset to collect biometric data.

Clause 125. The method of any of Clauses 123 to 124, wherein collecting the data about the child comprises using sensors on the VR headset to collect biometric data in real-time during administration of the first and second gamified visual tests.

Clause 126. The method of any of Clauses 123 to 125, further comprising processing the data to evaluate an engagement level of the child.

Clause 127. The method of any of Clauses 123 to 126, further comprising processing the data to evaluate a stress level of the child.

Clause 128. The method of any of Clauses 123 to 127, wherein collecting the first and second input comprises collecting the first and second input in real-time during administration of the first and second gamified visual tests.

Clause 129. The method of any of Clauses 123 to 128, further comprising processing the first and second input, wherein processing the first and second input comprises evaluating the child's visual health, an engagement level of the child, or a stress level of the child.

Clause 130. The method of any of Clauses 123 to 129, further comprising processing the first and second input in real-time during administration of the first and second gamified visual tests.

Clause 131. The method of any of Clauses 123 to 130, further comprising implementing an artificial intelligence to process the first and second input.

Clause 132. The method of any of Clauses 123 to 131, wherein the second gamified visual test comprises a different difficulty than the first gamified visual test and the method further comprises implementing an artificial intelligence to determine the different difficulty of the second gamified visual test.

Clause 133. The method of any of Clauses 123 to 132, wherein implementing an artificial intelligence to determine the different difficulty of the second gamified visual test comprises tailoring the different difficulty of the second gamified visual test to the child based on the child's visual health, an engagement level of the child, or a stress level of the child.

Clause 134. The method of any of Clauses 123 to 133, wherein implementing an artificial intelligence to determine the different difficulty of the second gamified visual test further comprises tailoring the different difficulty of the second gamified visual test to the child based on an age or development stage of the child.

Clause 135. The method of any of Clauses 123 to 134, wherein the second gamified visual test comprises a different nature than the first gamified visual test and the method further comprises implementing an artificial intelligence to determine the different nature of the second gamified visual test.

Clause 136. The method of any of Clauses 123 to 135, wherein implementing an artificial intelligence to determine the different nature of the second gamified visual test comprises tailoring the different nature of the second gamified visual test to the child based on the child's visual health, an engagement level of the child, or a stress level of the child.

Clause 137. The method of any of Clauses 123 to 136, wherein implementing an artificial intelligence to determine the different nature of the second gamified visual test further comprises tailoring the different nature of the second gamified visual test to the child based on an age or development stage of the child.

Clause 138. The method of any of Clauses 123 to 137, further comprising implementing an artificial intelligence to process the second input.

Clause 139. The method of any of Clauses 123 to 138, wherein evaluating the child's visual health comprises comparing at least one of the first or second input with a database.

Clause 140. The method of any of Clauses 123 to 139, wherein evaluating the child's visual health comprises comparing the first input with a database.

Clause 141. The method of any of Clauses 123 to 140, wherein evaluating the child's visual health comprises comparing the second input with a database.

Clause 142. A system for conducting children's vision screening, the system comprising: a virtual reality (VR) headset having a screen and at least one sensor configured to collect data about or input from a child wearing the VR headset; and a computing device in electronic communication with the VR headset and being configured to process the data or input from the patient and control an environment displayed on the screen based on the data or input, wherein the computing device is configured to (a) administer a first gamified visual test on the screen and receive a first input from the VR headset in response to the first gamified visual test, (b) administer a second gamified visual test on the screen and receive a second input from the VR headset in response to the second gamified visual test, and (c) compare at least one of the first or second input with a database for evaluating a child's visual health.

Clause 143. The system of Clause 142, wherein the data comprises visual responses identified by the at least one sensor, the visual responses comprising a child's eye movements or blink rates.

Clause 144. The system of any of Clauses 142 to 143, further comprising a microphone positioned on the VR headset and a handheld device in electronic communication with the VR headset and wherein the data or input comprise audio responses identified by the microphone or tactile responses identified by the sensors or the handheld device.

Clause 145. The system of any of Clauses 142 to 144, wherein the first and second input comprise voluntary and involuntary responses by the child to the first and second gamified visual tests.

Clause 146. The system of any of Clauses 142 to 145, wherein the at least one sensor is configured to collect the first and second input.

Clause 147. The system of any of Clauses 142 to 146, wherein the computing device is configured to compare the first and second input to the database for evaluating the child's visual health.

Clause 148. The system of any of Clauses 142 to 147, wherein the computing device is configured to compare the first input to the database for evaluating the child's visual health.

Clause 149. The system of any of Clauses 142 to 148, wherein the computing device is configured to compare the second input to the database for evaluating the child's visual health.

Clause 150. A method for tracking a child's visual health, the method comprising: collecting data about or input from a child wearing a virtual reality (VR) headset; administering a first gamified visual test on a screen of the VR headset; collecting a first input regarding a response from the child to the first gamified visual test; administering a second gamified visual test on the screen; collecting a second input regarding a response from the child to the second gamified visual test; producing a report; and receiving input from a third party, wherein the input redirects a difficulty or nature of the second gamified visual test.

Clause 151. The method of Clause 150, wherein collecting the data about the child comprises using sensors on the VR headset to collect biometric data.

Clause 152. The method of any of Clauses 150 to 151, wherein collecting the data about the child comprises using sensors on the VR headset to collect biometric data in real-time during administration of the first and second gamified visual tests.

Clause 153. The method of any of Clauses 150 to 152, further comprising processing the data to evaluate an engagement level of the child.

Clause 154. The method of any of Clauses 150 to 153, further comprising processing the data to evaluate a stress level of the child.

Clause 155. The method of any of Clauses 150 to 154, wherein collecting the first and second input comprises collecting the first and second input in real-time during administration of the first and second gamified visual tests.

Clause 156. The method of any of Clauses 150 to 155, further comprising processing the first and second input, wherein processing the first and second input comprises evaluating the child's visual health, an engagement level of the child, or a stress level of the child.

Clause 157. The method of any of Clauses 150 to 156, further comprising processing the first and second input in real-time during administration of the first and second gamified visual tests.

Clause 158. The method of any of Clauses 150 to 157, further comprising implementing an artificial intelligence to process the first and second input.

Clause 159. The method of any of Clauses 150 to 158, wherein the second gamified visual test comprises a different difficulty than the first gamified visual test and the method further comprises implementing an artificial intelligence to determine the different difficulty of the second gamified visual test.

Clause 160. The method of any of Clauses 150 to 159, wherein implementing an artificial intelligence to determine the different difficulty of the second gamified visual test comprises tailoring the different difficulty of the second gamified visual test to the child based on the child's visual health, an engagement level of the child, or a stress level of the child.

Clause 161. The method of any of Clauses 150 to 160, wherein implementing an artificial intelligence to determine the different difficulty of the second gamified visual test further comprises tailoring the different difficulty of the second gamified visual test to the child based on an age or development stage of the child.

Clause 162. The method of any of Clauses 150 to 161, wherein the second gamified visual test comprises a different nature than the first gamified visual test and the method further comprises implementing an artificial intelligence to determine the different nature of the second gamified visual test.

Clause 163. The method of any of Clauses 150 to 162, wherein implementing an artificial intelligence to determine the different nature of the second gamified visual test comprises tailoring the different nature of the second gamified visual test to the child based on the child's visual health, an engagement level of the child, or a stress level of the child.

Clause 164. The method of any of Clauses 150 to 163, wherein implementing an artificial intelligence to determine the different nature of the second gamified visual test further comprises tailoring the different nature of the second gamified visual test to the child based on an age or development stage of the child.

Clause 165. The method of any of Clauses 150 to 164, further comprising implementing an artificial intelligence to process the second input.

Clause 166. The method of any of Clauses 150 to 165, wherein producing the report comprises producing the report in real time.

Clause 167. The method of any of Clauses 150 to 166, wherein producing the report comprises producing a summary of the child's visual health, recommendations for further testing, or recommendations for corrective measures.

Clause 168. An interactive system for tracking a child's visual health, the system comprising: a virtual reality (VR) headset having a screen and at least one sensor configured to collect data about or input from a child wearing the VR headset; a computing device in electronic communication with the VR headset and being configured to process the data or input from the child and control an environment displayed on the screen based on the data or input, wherein the computing device is configured to (a) administer a first gamified visual test on the screen and receive a first input from the VR headset in response to the first gamified visual test, (b) administer a second gamified visual test on the screen and receive a second input from the VR headset in response to the second gamified visual test, and (c) produce a report; and a user interface configured to (a) display the report; and (b) receive input from a third party, wherein the input redirects a difficulty or nature of the second gamified visual test.

Clause 169. The system of Clause 168, wherein the data comprises visual responses identified by the at least one sensor, the visual responses comprising a child's eye movements or blink rates.

Clause 170. The system of any of Clauses 168 to 169, further comprising a microphone positioned on the VR headset and a handheld device in electronic communication with the VR headset and wherein the data or input comprise audio responses identified by the microphone or tactile responses identified by the sensors or the handheld device.

Clause 171. The system of any of Clauses 168 to 170, wherein the first and second input comprise voluntary and involuntary responses by the child to the first and second gamified visual tests.

Clause 172. The system of any of Clauses 168 to 171, wherein the at least one sensor is configured to collect the first and second input.

Clause 173. The system of any of Clauses 168 to 172, wherein the computing device is configured to compare the first and second input to a database for evaluating the child's visual health.

Clause 174. The system of any of Clauses 168 to 173, wherein the third party is a physician or parent.

Clause 175. A method for detecting visual processing disorders, the method comprising: collecting data about or input from a patient wearing a virtual reality (VR) headset; producing an environment having visual, auditory, and tactile stimuli; conducting a visual task; collecting a first input from the patient in response to the visual task; identifying discrepancies in the first input; reconducting the visual task; collecting a second input; and comparing at least one of the first or second input with a database to evaluate the patient for visual processing disorders.

Clause 176. The method of Clause 175, wherein the VR headset comprises sensors, cameras, or microphones, and collecting a first input comprises using the sensors, the cameras, or the microphones to monitor one or more of a patient's eye movements, reaction times, and coordination.

Clause 177. The method of any of Clauses 175 to 176, wherein reconducting the visual task further comprises providing feedback to the patient.

Clause 178. The method of any of Clauses 175 to 177, wherein the feedback comprises auditory stimuli.

Clause 179. The method of any of Clauses 175 to 178, wherein the feedback comprises tactile stimuli.

Clause 180. The method of any of Clauses 175 to 179, wherein the feedback comprises adjustments to an intensity or nature of the visual, auditory, and tactile stimuli of the environment.

Clause 181. The method of any of Clauses 175 to 180, further comprising implementing an adaptation algorithm to determine the intensity or nature of the visual, auditory, and tactile stimuli of the environment.

Clause 182. The method of any of Clauses 175 to 181, wherein providing the feedback comprises providing the feedback in real-time.

Clause 183. The method of any of Clauses 175 to 182, wherein providing the feedback comprises providing the feedback while reconducting the visual task.

Clause 184. The method of any of Clauses 175 to 183, wherein providing the feedback comprises informing the patient that the visual task requires the second input to ensure that the patient remains engaged with the visual task.

Clause 185. The method of any of Clauses 175 to 184, wherein collecting the first and second input comprises implementing an adaptation algorithm to process the first and second input to identify discrepancies.

Clause 186. The method of any of Clauses 175 to 185, wherein comparing comprises implementing an adaptation algorithm to compare at least one of the first or second input with a database.

Clause 187. The method of any of Clauses 175 to 186, wherein comparing comprises comparing at least one of the first or second input with a database to evaluate the patient for visual processing disorders.

Clause 188. The method of any of Clauses 175 to 187, wherein comparing comprises comparing the first input with a database to evaluate the patient for visual processing disorders.

Clause 189. The method of any of Clauses 175 to 188, wherein comparing comprises comparing the second input with a database to evaluate the patient for visual processing disorders.

Clause 190. The method of any of Clauses 175 to 189, further comprising recommending treatment based on a severity or likelihood of a patient's visual processing disorders.

Clause 191. A system for detecting visual processing disorders, the system comprising: a virtual reality (VR) headset having at least one sensor configured to collect data about or input from a patient wearing the VR headset; and a computing device in electronic communication with the VR headset and being configured to process the data or input from the patient and control an environment having visual, auditory, and tactile stimuli emitted from the VR headset based on the data or input, wherein the computing device is configured to (a) conduct a visual task through the VR headset and receive a first input from the patient in response to the visual task, (b) identify discrepancies in the first input, (c) reconduct the visual task and receive a second input from the patient in response to the visual task, and (d) compare at least one of the first or second input with a database to evaluate the patient for visual processing disorders.

Clause 192. The system of Clause 191, wherein the VR headset comprises one or more of a screen, vibrating motors, cameras, speakers, and microphones.

Clause 193. The system of any of Clauses 191 to 192, further comprising a handheld device in electronic communication with the VR headset and the computing device and having vibrating motors or sensors.

Clause 194. The system of any of Clauses 191 to 193, wherein the first and second input comprise one or more of visual input received at the sensors or the cameras, audio input received at the microphones, and tactile input received at the handheld device.

Clause 195. The VR system of any of Clauses 191 to 194, wherein the screen is configured to provide visual stimulus, the speakers are configured to provide auditory stimulus, and the vibrating motors are configured to provide tactile stimulus.

Clause 196. The system of any of Clauses 191 to 195, wherein the data comprises a patient's physiological responses during the visual task, wherein physiological responses comprise one or more of pupil dilation, blink rates, heart rate, and skin conductance.

Clause 197. The system of any of Clauses 191 to 196, wherein the computing device is configured to compare the first and second input to the database to evaluate the patient for visual processing disorders.

Clause 198. The system of any of Clauses 191 to 197, wherein the computing device is configured to compare the first input to the database to evaluate the patient for visual processing disorders.

Clause 199. The system of any of Clauses 191 to 198, wherein the computing device is configured to compare the second input to the database to evaluate the patient for visual processing disorders.

Clause 200. A method for treating visual processing disorders, the method comprising: collecting data about or input from a patient wearing a virtual reality (VR) headset; producing an environment having visual, auditory, and tactile stimuli; conducting a first visual task; collecting a first input from the patient in response to the visual task; determining a severity of a patient's visual processing disorder; conducting a second visual task; and collecting a second input.

Clause 201. The method of Clause 200, further comprising implementing an adaptation algorithm to evaluate the first input and determine the severity of the patient's visual processing disorder.

Clause 202. The method of any of Clauses 200 to 201, wherein the second visual task comprises a different difficulty than the first visual task.

Clause 203. The method of any of Clauses 200 to 202, further comprising implementing an adaptation algorithm to evaluate the first input and determine the different difficulty of the second visual task.

Clause 204. The method of any of Clauses 200 to 203, further comprising implementing an adaptation algorithm to evaluate the first input and determine the different difficulty of the second visual task in real-time.

Clause 205. The method of any of Clauses 200 to 204, further comprising implementing an adaptation algorithm to tailor the different difficulty of the second visual task to the patient based on the severity of the patient's visual processing disorder.

Clause 206. The method of any of Clauses 200 to 205, wherein the second visual task comprises a different nature than the first visual task.

Clause 207. The method of any of Clauses 200 to 206, further comprising implementing an adaptation algorithm to evaluate the first input and determine the different nature of the second visual task.

Clause 208. The method of any of Clauses 200 to 207, further comprising implementing an adaptation algorithm to evaluate the first input and determine the different nature of the second visual task in real-time.

Clause 209. The method of any of Clauses 200 to 208, further comprising implementing an adaptation algorithm to tailor the different nature of the second visual task to the patient based on the severity of the patient's visual processing disorder.

Clause 210. The method of any of Clauses 200 to 209, wherein conducting the second visual task comprises adjusting an intensity of the visual, auditory, and tactile stimuli.

Clause 211. The method of any of Clauses 200 to 210, wherein conducting the second visual task comprises implementing an adaptation algorithm to adjust an intensity or nature of the visual, auditory, and tactile stimuli.

Clause 212. The method of any of Clauses 200 to 211, wherein conducting the second visual task comprises adjusting a nature of the visual, auditory, and tactile stimuli.

Clause 213. The method of any of Clauses 200 to 212, wherein conducting the second visual task comprises implementing an adaptation algorithm to adjust an intensity or nature of the visual, auditory, and tactile stimuli.

Clause 214. A system for treating visual processing disorders, the system comprising: a virtual reality (VR) headset having at least one sensor configured to collect data about or input from a patient wearing the VR headset; and a computing device in electronic communication with the VR headset and being configured to process the data or input from the patient and control an environment having visual, auditory, and tactile stimuli emitted from the VR headset based on the data or input, wherein the computing device is configured to (a) conduct a first visual task through the VR headset and receive a first input from the patient in response to the visual task, (b) determine a severity of a patient's visual processing disorder, and (c) conduct a second visual task through the VR headset and receive a second input from the patient in response to the second visual task.

Clause 215. The system of Clause 214, wherein the VR headset comprises one or more of a screen, vibrating motors, cameras, speakers, and microphones.

Clause 216. The system of any of Clauses 214 to 215, further comprising a handheld device in electronic communication with the VR headset and the computing device and having vibrating motors or sensors.

Clause 217. The system of any of Clauses 214 to 216, wherein the first and second input comprise one or more of visual input received at the sensors or the cameras, audio input received at the microphones, and tactile input received at the handheld device.

Clause 218. The VR system of any of Clauses 214 to 217, wherein the screen is configured to provide visual stimulus, the speakers are configured to provide auditory stimulus, and the vibrating motors are configured to provide tactile stimulus.

Clause 219. The system of any of Clauses 214 to 218, wherein the data comprises a patient's physiological responses during the visual task, wherein physiological responses comprise one or more of pupil dilation, blink rates, heart rate, and skin conductance.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Furthermore, to the extent that the term “include,” “have,” or the like is used in the description or the claims, such term is intended to be inclusive in a manner similar to the term “comprise” as “comprise” is interpreted when employed as a transitional word in a claim.

As used herein, the term “about” is relative to the actual value stated, as will be appreciated by those of skill in the art, and allows for approximations, inaccuracies, and limits of measurement under the relevant circumstances. In one or more aspects, the terms “about,” “substantially,” and “approximately” may provide an industry-accepted tolerance for their corresponding terms and/or relativity between items.

As used herein, the term “comprising” indicates the presence of the specified integer(s), but allows for the possibility of other integers, unspecified. This term does not imply any particular proportion of the specified integers. Variations of the word “comprising,” such as “comprise” and “comprises,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. A method for detecting eye misalignment, the method comprising:
conducting a first visual task on a screen of a virtual reality (VR) headset worn by a patient;
collecting a first input regarding a response from the patient to the visual task;
conducting a second visual task on the screen;
collecting a second input regarding the response from the patient to the second visual task, wherein the second visual task comprises a different difficulty or nature than the first visual task;
based on at least one of the first or second input, evaluating the patient's eye coordination to determine whether the patient has eye misalignment.

2. The method of claim 1, further comprising implementing a multi-layered algorithm to process the first and second input and evaluate an eye coordination of the patient.

3. The method of claim 2, wherein the multi-layered algorithm processes the first and second input and evaluates the eye coordination in real-time.

4. The method of claim 1, further comprising implementing a multi-layered algorithm to determine the different difficulty of the second visual task.

5. The method of claim 1, further comprising implementing a multi-layered algorithm to determine the different nature of the second visual task.

6. The method of claim 1, wherein evaluating the patient's eye coordination comprises determining a degree of eye misalignment.

7. The method of claim 1, further comprising diagnosing the patient with eye misalignment.

8. The method of claim 7, wherein diagnosing the patient with eye misalignment comprises implementing a multi-layered algorithm to compare the first or second input to a database to evaluate the patient for eye misalignment.

9. A method for correcting eye misalignment, the method comprising:
conducting a first visual task on a screen of a virtual reality (VR) headset worn by a patient;
collecting a first input regarding a response from the patient to the visual task;
providing corrective feedback to the patient;
determining a severity and probability of future eye misalignment for the patient; and
conducting a second visual task on the screen, wherein the second visual task comprises a different difficulty or nature than the first visual task.

10. The method of claim 9, wherein the method further comprises, before conducting the first visual task, receiving a medical history and biodata of the patient.

11. The method of claim 9, wherein conducting the first and second visual tasks comprises strengthening binocular vision and preventing progression of misalignment.

12. The method of claim 9, wherein providing corrective feedback comprises providing visual stimuli on the screen.

13. The method of claim 9, wherein providing corrective feedback comprises providing haptic stimuli at vibrating motors positioned on the VR headset.

14. The method of claim 9, wherein providing corrective feedback comprises providing auditory stimuli on the speakers positioned on the VR headset.

15. The method of claim 9, further comprising implementing a multi-layered algorithm to process the first and second input and evaluate an eye coordination of the patient.

16. The method of claim 15, wherein the multi-layered algorithm processes the first and second input and evaluates the eye coordination in real-time.

17. The method of claim 9, further comprising implementing a multi-layered algorithm to determine the different difficulty of the second visual task.

18. The method of claim 17, wherein the multi-layered algorithm determines the different difficulty of the second visual task in real-time.

19. The method of claim 9, further comprising implementing a multi-layered algorithm to determine the different nature of the second visual task.

20. The method of claim 19, wherein the multi-layered algorithm determines the different nature of the second visual task in real-time.
